SEQUENCE LISTING (3 sequences)

SEQ ID NO: 1 - DNA, 1016 bp, synthetic construct
  source 1..1016: mol_type = other DNA
aaataaataaataaataaataaataaataaataaataaataaataaataaataaatacacacacacacacacacacacacacacacacacacacacacacacacacacacacacacgggtgggtgggtgggtgggtgggtgggtgggtgggtgggtgggtgggtgggtgggtgggtgggtgggtgggtgggtgggtgggtgggtgggtgggtgggtgggtgggtgggtgtaaagtaaagtaaagtaaagtaaagtaaagtaaagtaaagtaaagtaaagtaaagtaaagtaaagtaaagtaaagtaaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtttgtttgtttgtttgtttgtttgtttgtttgtttgtttgtttgtttgtttgtttgtttgtttgtttgtttgtttgtttgtttgtttgtttgtttgtttgtttgtttgttt

SEQ ID NO: 2 - DNA, 1182 bp, synthetic construct
  source 1..1182: mol_type = other DNA
aaataaataaataaataaataaataaataaataaataaataaataaataaataaatacacacacacacacacacacacacacacacacacacacacacacacacacacacacacacgggtgggtgggtgggtgggtgggtgggtgggtgggtgggtgggtgggtgggtgggtgggtgggtgggtgggtgggtgggtgggtgggtgggtgggtgggtgggtgggtgggtgggtgtgggtgggtgtgggtgtaaagtaaagtaaagtaaagtaaagtaaagtaaagtaaagtaaagtaaagtaaagtaaagtaaagtaaagtaaagtaaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtaagtccccccgtgtccccccgtgtccgtccgtgtccgtccgtgtcgtcgtcgtcgtcgtcgtgggtgggtgtgggtgggtgtgtccgtgtgtgtccgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtttgtttgtttgtttgtttgtttgtttgtttgtttgtttgtttgtttgtttgtttgtttgtttgtttgtttgtttgtttgtttgtttgtttgtttgtttgtttgtttgttt

SEQ ID NO: 3 - DNA, 4943 bp, synthetic construct
  source 1..4943: mol_type = other DNA
ggggccggttccccttaattaaggggaaccaaggggaaggccggccggggccaaaaaaaaccggttttggggggggttccaattccccttccggccccccccccggttggttggttttaattccggccaaggggggttccccaattccttttggttaattccccggggggccggaaaattccggccttccccaaggaattttttccaaaaaattggttttaaggttggttggggttggggttttccggccttccggccttaaaaaattaaggccaaaaaattaaccttggttttaaaaccaaggggggttaaaaggttttccggaaccccttggttaaccaattggaaccggttaaccggccggaaggttttaaggccggaaggttggccggaattccggggggccccggggttaaccaaaaccttttttttccaaaaaattggccccggggttttccttggggttccaattttccggggggaaccaaggccggggttaaccaaaattaaccccccttggccggggttttccggccaaggaattccggttccggaaaattttggaaccaaggccggttaaaattttaaggggttggaaccggaaggggggccggttaattggttttggccggaaggccaattttccggggggttttccccccggttggccggttccccggaaccccaaggttggaaccttttggaattttttggggaaaaccttggccggttccggccggccttccggaattggttccggggccggaaaattggggttttaaccccaattaaggttaaggccccccccaaggaattaaggaaccccccggaaggccaaccccggttaaaaccggccttggggaaaaggggggttaaggggttaattggccccttccggttttttttccggggccttccaaaaaaccaaggccggttggccttggaattggttttaaggaattggttccccaaccaaggttggaaccggaaaaaaccaaggccaaaaaaggaaaaaaaaggccccccttccaaaaaaggggaattccttggccttggggccttggttccggttttaattccaaccggccccggaaccaaccggggttttccggggaaaattttttttccaattttttaattaaaattccggaaggaaggggggaaccaaccccaaccggttccttaattggaaccttccccggggaaaaggccggaaggccaattccggggaaggggttggaattggggccaaggggccggccttccaaccaattaaaaggttggaattttggggaattttccccggttccccaattggttccaaggttccggggccaaaattggttggaaggttaattccttggccccggttggggttccaaaaggggaaccttaattaaggttccaaccccccccttaaaaggaaggccaattaaggaaggggggaattttggttggaattaaaaggccaaggccttccttaaaaaaaaaattaaggttttttggttaaggttaattaaaaggggccaaaaggaaaaaaccccccttccttccccaattccggaaggggttttttccccggggccttggccttggccccccaattttaaggaaaaaaaattttggttaaggttaattttccttccccaaaattttttccaattggaaggtcatgccatccttgagggattcacactgcggtaacctgccggcaagatagcctacaacaggcacgcgttgaaatggatctccttagaatatggacttcgtgtcccccaagaaccattgagctattacatcttctggctcagggtcattatactcccaatcctgcgctcgtatattctgcgcgatccatacgtgcactgatttgtgtaactatcaagtgataccctccccgacaacttcgtctggtccgttagctccccccgaaaatttgcgggaaaccgttccctactagatctctcctatttagtggtggtaggtctggaggcgcaatcgtcaaactataagcgccgggagcgctgatgcgccaacccgggcgcaagtgaccactataaaaaactctctatggtacctagaaaaccagccaggggcataagttttcagcgttaactgagccgtttggaatacgagcatcggaggtgatgggcaggcgctcacataagtgattggattccgtccaatgtcagtccggcaagtgagtatcttgccgtggtcacggactatagtcacccctaagagcgatagagggatgtgataagccagctctaaaaatagtttgtagtataaggcaatgaaaccctctcacatcgagttttccggctgctggcccattagaaaattgtaggtatctccaatttcatgagctcatgccaccttgagggattcacactgcggtcaactgccggcaagatagcctacaacaggcaccgcgtgaaatggatctccttagaatattgacttcgtgtcccccaagaacgcattgagctattacatttctggctcagggtcattaatactcccaacctgcgctacgtatattctgcggactccatacgtgcactgatttgtgtaactccccctgcgggacgtccactgaagttcgctgtctgtgctttcaggcgttgccaaggccttcgccaggcctgtgcctcaccctctgcattggatcacggcagcgaagagagatgacggtagacttcccatgtggtgaccccattggtatttaacacttttgcttagattatctcggcgtcgcaattggggtgacgcggagaaaccaggcgggctggtgtcgatcgacaactgcgctcgcggttcactctctaaacgtaagttcagccgtccggctagtggtgtgcaatgggtacacggacctgtgcaaccttgcgtgaagcgcgcttaaagatgttaagggagggtgaggttcagcgacttctctgaaacctgtgcccctgagacacgtgtttcacctgtagatggaggtatggcaactgcatttacctttttctgaggcagacctgtacctcgagtgcttaatggtctcagtggccgtgggtctcttgacaatgtctggtacggggttccctgagacggccgggctgagttcattaatagaatccatacggaagagcccacttatcagcgagtaacagaggccgcacaccgatttgtctgggtctctagtcgagatctggggcacaatcctttacttaagatctgatcttccctactatggatatgtccttaggatacggtacttaccggggcgcgggagcaaccatgatcagcagtatctgaacattgccgaacctcgtattttagttctcttcgcacattcggaacgcggttcaattaggtgtagaggtcccataatggacaggcccaagtagatccgccaggcgatgcttgtagcccatcgcgcagctcgcggtttcatggaatccctcccttgcaccagcgacgagcaccgggttggaccgtatgctcttctggccgtggaaagtcaaccggatatcctcggtgaataaagaggaacccatcgaaggatcttattgctgtgtagcgcgactttcaaacgcccaggagttacacgtcgtgacaatgacctgcatatcagattgaggcatgtagccccctatattcactagctcaatacggcccgcaaagcaagggttactgagcaactgcctagtacgtctataagcaagattccggtattcgtataaggctaaagtttgtgcggtcttaggaaatcgtaatgcacaacgactgacggaaggacgataaggacatggctccatctttgcgctttactcatgtaccaagggcggcctggcatgtggggctgcagttctggcggcaatgggttcccaccccaagggttggtcatggctacaacaagtgtgaaggcacccagtgaagcgacttttaagtgtagtctgggtagagaaaataaccaggcaccgtgtatattgctattcagtagccgctaatatacggtaatgtctgggttgtaactccagtcaacacactgtttggaaagcggtcaccccttacataggtgtcatggccattttgtaaacaatctataagcactacaaggaaggtcggtatatgcaactcccgccgtggtcggaggtcaccagcctcgtggcgtctgctctgtcgatgaaccaaagagacccccttggagatcttctcacggtcagggtacgctggttgctcccgaatagtactgaccacatcagagcatccaactaagtgccattctaaacagttgctcgaaatttgatgcttgtccgttagacagtctcccatcgtcaagtaaccgtccgattgaggttagctgagatcggccatttgggtaagaaatccgggaacatcgggaaaatggattcgtaacaacgcccccgcaggacgcaattcctaggagagaggtgagttactgttcaccgcattctaagccgtcgtcaaagcactttaacctggcatatgtacggggataagtctggatttagcctccttgcgagccgaattcggtctcccctattcgctgtgctctagtagtgatggaaggctaactttataatgtgcgtccttgaatcgcccgcacggatatcggtgattgttaacgcggctagtagataataggcgcgccaatagactatccattggagcggtcgtaggcgatcatatcaagagcttgtagtaagagaaataacgctgttgctcctagctggcgtcgactcgttgacctcacagtggttcagggagggtccgtttcacttgtttgaactccgcgacactgatggtcctgaaacctgtcgctgaccagggtgaacctgaggcactagactttgctcgataattgattctctgaggtcttgctttctcaagattgtcccctttgtcataggttacgctgcgcctcgtggaag

(19) United States
(12) Patent Application Publication
(10) Pub. No.: US 2024/0412824 A1
(43) Pub. Date: Dec. 12, 2024

(54) MACHINE LEARNING FOR THE DISCOVERY OF NANOMATERIAL-BASED MOLECULAR RECOGNITION

(71) Applicant: Massachusetts Institute of Technology, Cambridge, MA (US)

(72) Inventors: Xun Gong, Boston, MA (US); Michael Strano, Lexington, MA (US); Retsef Levi, Newton, MA (US); Nicholas Renegar, Cambridge, MA (US)

(21) Appl. No.: 18/689,059

(22) PCT Filed: Sep. 7, 2022
(86) PCT No.: PCT/US2022/042710

§ 371 Date: Mar. 4, 2024

Related U.S. Application Data
(60) Provisional application No. 63/241,123, filed on Sep. 7, 2021.

(51) Int. Cl. G16C 20/10; G06F 30/27; G16B 40/20; G16C 20/70

(52) U.S. Cl. CPC G16C 20/10; G06F 30/27; G16B 40/20; G16C 20/70

(57) ABSTRACT

Computer program products, computer systems, and computer-implemented methods for making and using computational models for prediction of molecular recognition (MR) between a nanomaterial (NM) MR binder and an analyte. Methods for making a computational model involve selecting a candidate NM MR binder and conducting a physical test to determine whether MR occurs between the candidate NM MR binder and an analyte, and correlating features of the candidate NM MR binder with an experimental result obtained from the physical test to produce predictive information for the computational model. Methods for using the computational model involve receiving features of an untested candidate NM MR binder and analyzing the features to produce a prediction score that represents an expected experimental result of a physical test of the untested candidate MR binder and associating the prediction score with the untested candidate MR binder.

### BACKGROUND

Molecular recognition (MR) occurs when a first molecule or atom binds or interacts with a second molecule or atom. These microscale events underpin a wide range of natural and artificial processes. A variety of molecules are capable of MR, including inorganic compounds, organic compounds, nanomaterials, small molecules, and large molecules such as biomolecules. Example biomolecules involved with MR include nucleotides (e.g., DNA, RNA), proteins (e.g., antibodies, enzymes), and lipids.

Biomolecular MR by antibodies and antibody engineering technologies have long been harnessed for commercial applications in medicine, diagnostic testing, and analytical applications. Existing approaches to generate antibody products for MR rely on B-lymphocyte biological processes, which while effective for some analytes in some instances, does not allow for the development of antibody products against analytes that either are not effective immunogens and cannot be processed by B-lymphocytes, or are otherwise not amenable to these approaches. Because of extensive reliance on existing antibody engineering techniques and the limited number of analytes targetable with antibodies, some analytes are effectively intractable to targeted MR.

In addition, analytes that are industrially targeted by antibodies are not usually targeted by other, experimental MR binders, due at least in part to the prevalence of risk averse business and investment practices. If the cost of development of alternative MR binders was lower, then the risk/benefit ratio would improve, and these approaches may become commercially viable and may complement or replace existing antibody engineering technologies.

Efforts to engineer targeted MR against analytes, whether with antibodies or with other types of molecules such as nucleotide aptamers, non-immunoglobulin protein scaffolds, molecularly imprinted polymers, and nanomaterials, are hindered by the vast size of the pool of possible molecular states and configurations as well as the lack of an intuitive framework for predicting the outcome of a particular design choice. The general unpredictability of whether an MR event would likely occur between a proposed MR binder and a given analyte necessitates physical testing of the proposed MR binder with the analyte to empirically determine whether MR occurs. This process is time-consuming and expensive.

Accordingly, there is a need for improved approaches for engineering targeted MR against analytes that reduce or eliminate the requirement for physical testing of a large number of candidate MR binders for a given analyte. The present invention meets this and other long-felt and unmet needs.

### SUMMARY

In general, the disclosure provides computer-implemented methods for making and using computational machine learning (ML) models for prediction of MR between a candidate nanomaterial MR binder and an analyte of interest. These approaches involve generating a list of candidate MR binders and analyzing features of and physically testing a subset of the total number of candidate MR binders, and using the results from physical testing of the subset to train a ML model to predict the likelihood of MR occurring between the analyte and untested candidate MR binders that share the same or similar features with the tested candidate MR binders.

The disclosed approaches, while implemented with artificial intelligence (AI) and/or ML computational techniques, are grounded in laboratory test results, which allows the ML model to improve with additional testing and provides a reliable predictive framework for intelligent design of MR binders without the need for exhaustively testing every candidate MR binder. This enables MR designers to focus on validating those candidate MR binders that are predicted by the model to have a higher likelihood of successful MR. As a result, targeted MR can be designed more efficiently, and this opens up possibilities for the development of a wider variety of MR binders for existing analytes and also increases the variety of analytes that are tractable for targeted MR.

In one aspect, the disclosure provides a method, performed by at least one computer processor, of making a computational model stored on at least one non-transitory computer-readable medium. The method comprises selecting a candidate molecular recognition (MR) binder that comprises a nanomaterial, and in various embodiments, the nanomaterial is configured for signal transduction in response to a molecular recognition event that involves the candidate MR binder. The method also comprises recording an experimental result that corresponds with a physical test of the candidate MR binder, and correlating a feature of the candidate MR binder with the experimental result to produce predictive information that is embodied by the computational model. In embodiments, the method is performed by the at least one computer processor executing computer readable instructions stored on the at least one non-transitory computer-readable medium, and the processor may be embodied as part of a computational device or computer system.

In another aspect, the disclosure provides a method, performed by at least one computer processor, of using a computational model stored on at least one non-transitory computer-readable medium. The method comprises receiving, as an input, a feature of a candidate molecular recognition (MR) binder that comprises a nanomaterial, and again, in various embodiments, the nanomaterial is configured for signal transduction in response to a molecular recognition event that involves the candidate MR binder. The method of using the model also comprises analyzing the feature of the candidate MR binder, based on the computational model, to produce a prediction score that represents an expected experimental result of a physical test of the candidate MR binder, and associating the prediction score with the candidate MR binder. In embodiments, the method is performed by the at least one computer processor executing computer readable instructions stored on the at least one non-transitory computer-readable medium, and the processor may be embodied as part of a computational device or computer system.

In embodiments, the experimental result from physical testing is binary and indicates whether or not the molecular recognition event, which involves a physical interaction between the candidate MR binder and an analyte, occurred in the physical test. In embodiments, the experimental result indicates a degree to which the molecular recognition event occurred in the physical test, such that the experimental result may provide predictive information that is related to a continuum or gradient related to confidence of MR or, if MR is confidently predicted to occur, an expected property or characteristic of the MR, such as stability of an MR event.

In embodiments, the nanomaterial comprises a single-walled carbon nanotube (SWCNT) that is optically responsive to the molecular recognition event. The SWCNT may act as a scaffold for securing the interaction feature in place and also may act as a reporter for reporting binding events via optical responses or changes. An optical response may involve a visible and/or a non-visible optical change that is detectable by an optical detection system for integration of the methods of the disclosure with suitable laboratory equipment.

In embodiments, the candidate MR binder comprises an interaction feature for interaction with an analyte for the molecular recognition event. While the interaction feature may comprise any compound, chemical structure, or moiety, in particular embodiments, it comprises an aptamer, a polymer, a peptide, a polypeptide, a protein, a protein complex, a ribonucleic acid (RNA), or any combination thereof. In particular embodiments, the interaction feature comprises a polynucleotide, such as DNA, that is useful or potentially useful for MR with the analyte. While any analyte may be analyzed with a particular MR binder, in certain embodiments, the analyte is cadmium, enrofloxacin, chloramphenicol, semicarbazide, or any combination thereof.

In embodiments, the correlating the feature step of the methods implements ML techniques and may comprise analyzing, with a convolutional neural network (CNN), a local structure of the DNA (i.e., the interaction feature of the candidate MR binder) to make a local structure prediction (LSP), and may also comprise analyzing, with a principal components analysis (PCA), a high-level feature (HLF) of the DNA to make a high-level prediction (HLP). This step may also comprise analyzing, with a gradient-boosted decision tree (GBDT), the LSP and the HLP to make predictive information that relates the features of the candidate MR binder to the experimental result obtained from physical testing.

In other aspects, the disclosure provides one or more computer program products, such as one or more non-transitory computer-readable media, having stored thereon a computational model as disclosed herein.

In yet other aspects, the disclosure provides one or more computer devices or systems configured to perform a method as disclosed herein.

Other features and advantages of various aspects and embodiments of the present invention will become apparent from the following description and from the claims.

### BRIEF DESCRIPTION OF THE DRAWINGS

FIG. 1 shows an overall, example experimental and computational scheme for data generation, including the collection, processing, modeling, and analysis of the photophysical response data from sensors.

FIG. 2 shows a flowchart of an example process for machine learning techniques for a predictive model of the present disclosure.

FIG. 3 shows a representation of an entire example dataset in the form of Pearson correlation coefficients between HLFs and sensor responses for the cases where the p-value <0.05.

FIG. 4 shows Pearson correlations between the predicted and actual sensor responses, and their corresponding p-values, with and without the local structure CNN predictions as model inputs.

FIG. 5 shows fitted model coefficients and statistical significance for an evaluation of significant features; a linear regression was fit for each experimental condition with HLF-PCs as independent features and the sensor response as the dependent feature, for all six experimental conditions from FIG. 4 with significant predictions at p<0.05.

FIG. 6 shows an assessment of the number of samples for predictive power under different experimental conditions.

FIG. 7 shows example sensor response function values.

FIG. 8 shows example final hyperparameters for Gradient-Boosted Decision Trees (GBDTs).

FIG. 9 shows an example of HLF-PC variance.

FIG. 10 shows an example of HLF-PC coefficients.

FIG. 11 shows changes in PL emission spectra associated with experimental conditions, highlighting variation based on analyte composition and CP structure.

FIG. 12 shows an example of raw PL emission changes.

FIG. 13 shows an example of sensor response curves for four cases ((a), (b), (c), and (d)).

FIG. 14 shows an example of HLF definitions.

FIG. 15 shows a flowchart of an example method for making a computational model, according to the disclosure.

FIG. 16 shows a flowchart of an example method for using a computational model, according to the disclosure.

FIG. 17 shows a flowchart of an example method for managing a computational model based on experimental results.

FIG. 18 shows a block diagram of a computer.

### DETAILED DESCRIPTION

The present disclosure provides computer-implemented methods for making and using computational machine learning (ML) models for prediction of molecular recognition (MR) between a candidate nanomaterial MR binder and an analyte of interest. The methods involve generating a list of candidate MR binders, analyzing features of candidate MR binders, and physically testing at least some of the candidate MR binders to produce test results. The results are used to train a ML model to predict the likelihood of MR occurring between the analyte and untested (out of sample) candidate MR binders that share the same or similar features with the tested candidate MR binders.

The methods are generally implemented with artificial intelligence (AI) and/or ML computational techniques that leverage laboratory test results for model training and improvement. The ML model may improve with additional testing, and may provide a reliable, predictive framework for intelligent design of MR binders without the need for exhaustively testing every candidate MR binder.

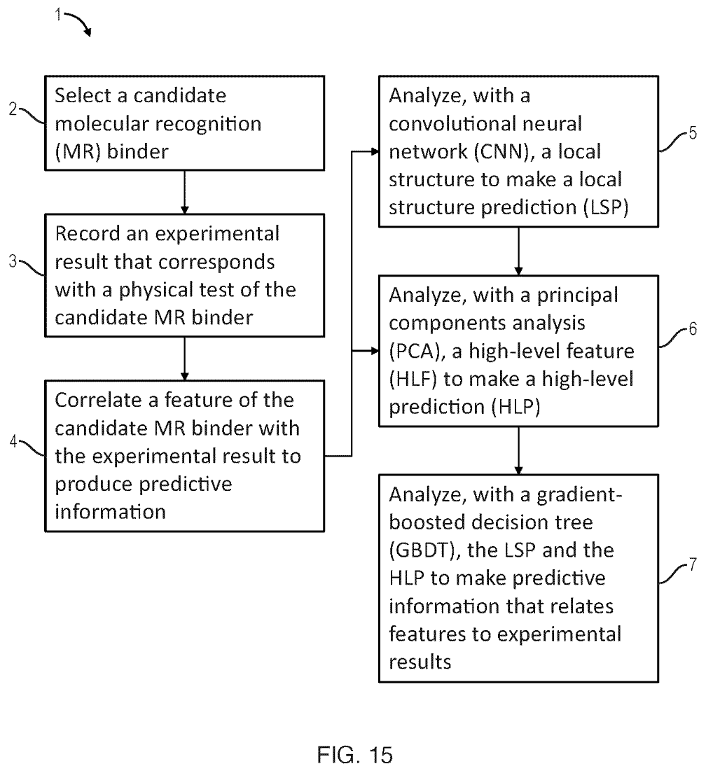

Referring now to FIG. 15, there is shown a flowchart of an example method for making a computational model, according to the disclosure. Generally, a method 1, performed by at least one computer processor, of making a computational model stored on at least one non-transitory computer-readable medium utilizes one or more computer processors and comprises selecting 2 a candidate molecular recognition (MR) binder that is to be physically tested and that comprises a nanomaterial.

While any type of nanomaterial may be used, in certain embodiments, a combination scaffold-signal nanomaterial is used. While two-component systems that utilize a scaffold component and a signal component separately may be used, a combination nanomaterial that acts as both scaffold and signaler may help to minimize interfacial losses that may occur with two-component designs, improving MR binder efficiency. Accordingly, in embodiments, the nanomaterial acts both as a scaffold for securing an interaction feature of the MR binder and as a signal transducer in response to a molecular recognition (MR) event that involves the candidate MR binder.

In embodiments, the method 1 of making the computational model also comprises recording 3 an experimental result that corresponds with a physical test of the candidate MR binder, and correlating 4 a feature of the candidate MR binder with the experimental result to produce predictive information, or predictive data, that is embodied by the computational model. The recording 3 the experimental result may involve, among other steps, automated or semi-automated or manual laboratory testing of the in-sample candidate MR binder, and the correlating 4 the feature may involve, among other steps, procedures for training the ML model. These procedures may include analyzing 5, with a convolutional neural network (CNN), a local structure of the in-sample candidate MR binder to make a local structure prediction (LSP) to be used for analysis and prediction for out-of-sample candidate MR binders. In addition to the LSP-generating procedures, or as an alternative, the analytical procedures may include analyzing 6, with a principal components analysis (PCA), a high-level feature (HLF) of the in-sample candidate MR binder to make a high-level prediction (HLP) to be used for analysis and prediction for out-of-sample candidate MR binders. In embodiments wherein the LSP and the HLP are both generated, the procedures may include analyzing 7, with a gradient-boosted decision tree (GBDT), the LSP and the HLP to make final predictive information or data that relates a plurality of features of the in-sample candidate MR binder to the experimental results. The method 1 may be performed by at least one computer processor executing computer readable instructions stored on the at least one non-transitory computer-readable medium, and the processor may be a part of a computational device or computer system.

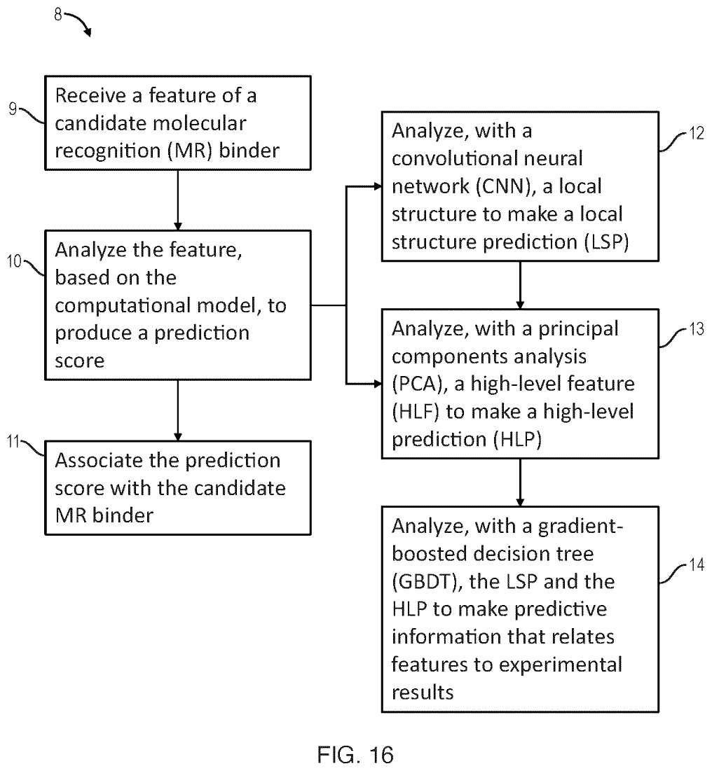

Referring now to FIG. 16, a method 8, performed by at least one computer processor, of using a computational model stored on at least one non-transitory computer-readable medium utilizes one or more computer processors and comprises receiving 9, as an input, a feature of an out-of-sample candidate molecular recognition (MR) binder that comprises a nanomaterial and is not to be or has not yet been validated with laboratory testing. The nanomaterial may be configured for both scaffolding and signal transduction. The method 8 also comprises analyzing 10 the feature of the out-of-sample candidate MR binder, based on the computational model, to produce a prediction score that represents an expected experimental result of a physical test of the out-of-sample candidate MR binder if it were to be physically tested in-sample, and associating 11 the prediction score with the out-of-sample candidate MR binder. The analyzing 10 the feature may involve, among other steps, procedures for applying the ML model. These procedures may include analyzing 12, with a convolutional neural network (CNN), a local structure of the out-of-sample candidate MR binder to use a local structure prediction (LSP) as to whether the out-of-sample candidate MR binder will effect a MR event with the analyte. In addition to the LSP-using procedures, or as an alternative, the procedures may include analyzing 13, with a principal components analysis (PCA), a high-level feature (HLF) of the out-of-sample candidate MR binder to use a high-level prediction (HLP) as to whether the out-of-sample candidate MR binder will effect a MR event with the analyte. In embodiments wherein the LSP and the HLP are both generated, the procedures may include analyzing 14, with a gradient-boosted decision tree (GBDT), the LSP and the HLP to use final predictive information or data that relates a plurality of features of the out-of-sample candidate MR binder to the predicted experimental results. In embodiments, the method 8 is performed by the at least one computer processor executing computer readable instructions stored on the at least one non-transitory computer-readable medium, and the processor may be embodied as part of a computational device or computer system.

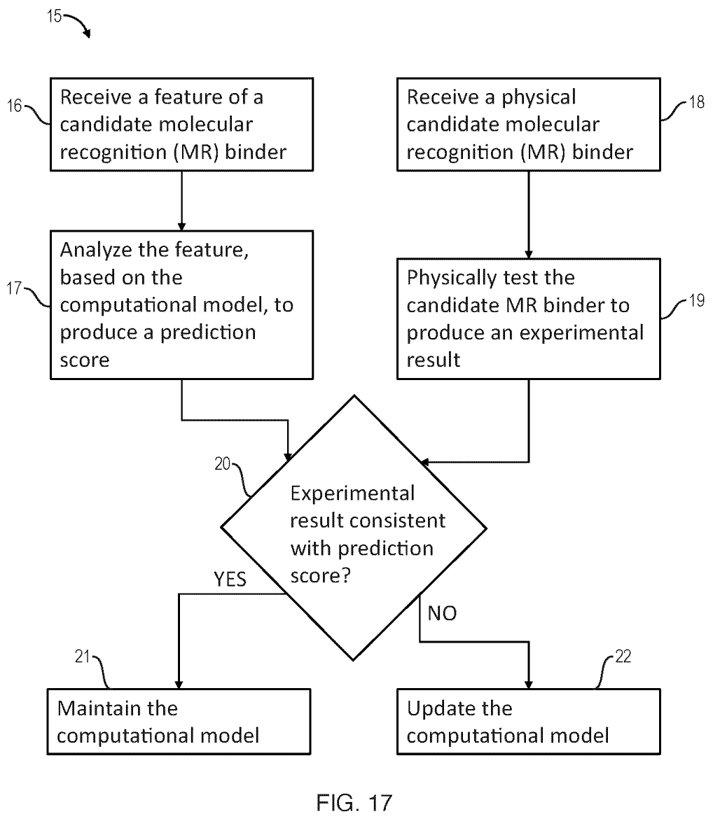

Referring now to FIG. 17, there is shown a flowchart of an example method for managing a computational model based on experimental results. The computational model may be maintained and updated by a method 15 that includes, among other possible steps, receiving 16 as information or data a feature of a candidate MR binder, analyzing 17 the feature with the computational model, receiving 18 as a physical material a candidate MR binder that corresponds to the candidate MR binder analyzed for the feature at step 17, and physically testing 19 the candidate MR binder to produce an experimental result. If the experimental result is consistent with a prediction score for the candidate MR binder (STEP 20: YES), then the method may involve no significant update to the computational model and may instead involve maintaining 21 the computational model in an approximately consistent state. However, if the experimental result is not consistent with the prediction score for the candidate MR binder (STEP 20: NO), then the method may involve updating 22 the computational model based on the experimental results. Accordingly, in various aspects of the disclosure, the computational model and the methods are generally grounded in and guided by truth resulting from physical testing.

In embodiments, the experimental result from physical testing is binary and indicates whether or not the molecular recognition event, which involves a physical interaction between the in-sample candidate MR binder and an analyte, occurred in the physical test. In embodiments, the experimental result indicates a degree to which the molecular recognition event occurred in the physical test, such that the experimental result may provide predictive information that is related to or positioned along a continuum or gradient related to confidence of MR or, if MR is confidently predicted to occur, an expected property or characteristic of the MR, such as stability of an MR binding event.

In embodiments, the nanomaterial comprises a single-walled carbon nanotube (SWCNT) that is optically responsive to the molecular recognition event. The SWCNT may act as a scaffold for securing the interaction feature in place and also may act as a reporter for reporting binding events via optical responses or changes. An optical response may involve a visible and/or a non-visible optical change that is detectable by an optical detection system for integration of the methods of the disclosure with suitable laboratory equipment.

In embodiments, the candidate MR binder comprises an interaction feature for interaction with an analyte for the molecular recognition event. While the interaction feature may comprise any compound, chemical structure, or moiety, in particular embodiments, it comprises an aptamer, a polymer, a peptide, a polypeptide, a protein, a protein complex, a ribonucleic acid (RNA), or any combination thereof. In certain embodiments, the interaction feature comprises a polynucleotide, such as DNA, that is useful or potentially useful for MR with the analyte. While any analyte may be analyzed with a particular MR binder, in certain embodiments, the analyte is cadmium, enrofloxacin, chloramphenicol, semicarbazide, or any combination thereof. Since analyte MR events may be able to produce distinctive signatures depending on the analyte, a sample may contain a plurality of analytes and a plurality of MR binders for independent detection and/or quantitation of analyte within the sample, improving practical application for testing of samples that are complex or heterogenous.

In embodiments, the disclosure provides one or more computer program products, such as one or more non-transitory computer-readable media, having stored thereon a computational model as disclosed herein. In yet other embodiments, the disclosure provides one or more computer devices or systems configured to perform a method as disclosed herein.

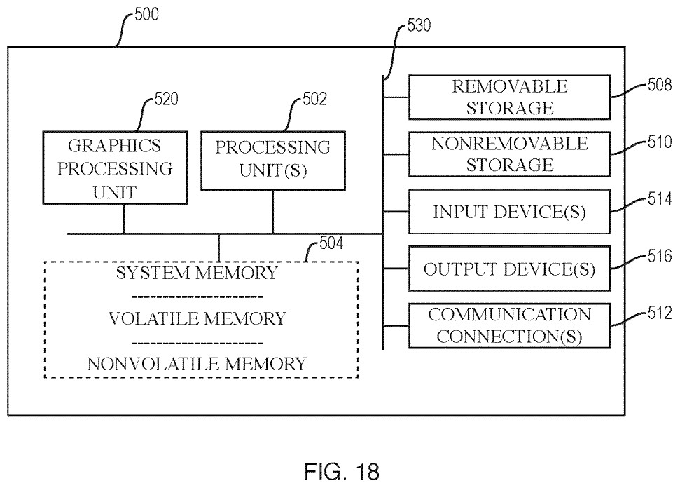

Referring now to FIG. 18, there is shown a block diagram of an example computer. The example computer processes computer programs using a processing system. Computer programs on a general-purpose computer generally include an operating system and applications. The operating system is a computer program running on the computer that manages access to resources of the computer by the applications and the operating system. The resources generally include memory, storage, communication interfaces, input devices and output devices.

Examples of such general-purpose computers include, but are not limited to, larger computer systems such as server computers, database computers, desktop computers, laptop and notebook computers, as well as mobile or handheld computing devices, such as a tablet computer, handheld computer, smart phone, media player, personal data assistant, audio and/or video recorder, or wearable computing device.

With reference to FIG. 18, an example computer 500 comprises a processing system including at least one processing unit 502 and a memory 504. The computer can have multiple processing units 502 and multiple devices implementing the memory 504. A processing unit 502 can include one or more processing cores (not shown) that operate independently of each other. Additional co-processing units, such as graphics processing unit 520, also can be present in the computer. The memory 504 may include volatile devices (such as dynamic random-access memory (DRAM) or other random-access memory device), and non-volatile devices (such as a read-only memory, flash memory, and the like) or some combination of the two, and optionally including any memory available in a processing device. Other memory such as dedicated memory or registers also can reside in a processing unit. Such a memory configures is delineated by the dashed line 504 in FIG. 18. The computer 500 may include additional storage (removable and/or non-removable) including, but not limited to, solid state devices, or magnetically recorded or optically recorded disks or tape. Such additional storage is illustrated in FIG. 18 by removable storage 508 and non-removable storage 510.

The various components in FIG. 18 are generally interconnected by an interconnection mechanism, such as one or more buses 530. A computer storage medium is any medium in which data can be stored in and retrieved from addressable physical storage locations by the computer. Computer storage media includes volatile and nonvolatile memory devices, and removable and non-removable storage devices. Memory 504, removable storage 508 and non-removable storage 510 are all examples of computer storage media. Some examples of computer storage media are RAM, ROM, EEPROM, flash memory or other memory technology, CD-ROM, digital versatile disks (DVD) or other optically or magneto-optically recorded storage device, magnetic cassettes, magnetic tape, magnetic disk storage or other magnetic storage devices. Computer storage media and communication media are mutually exclusive categories of media. The computer 500 may also include communications connection(s) 512 that allow the computer to communicate with other devices over a communication medium. Communication media typically transmit computer program code, data structures, program modules or other data over a wired or wireless substance by propagating a modulated data signal such as a carrier wave or other transport mechanism over the substance. The term “modulated data signal” means a signal that has one or more of its characteristics set or changed in such a manner as to encode information in the signal, thereby changing the configuration or state of the receiving device of the signal. By way of example, and not limitation, communication media includes wired media such as a wired network or direct-wired connection, and wireless media include any non-wired communication media that allows propagation of signals, such as acoustic, electromagnetic, electrical, optical, infrared, radio frequency and other signals.

Communications connections 512 are devices, such as a network interface or radio transmitter, that interface with the communication media to transmit data over and receive data from signals propagated through communication media. The communications connections can include one or more radio transmitters for telephonic communications over cellular telephone networks, and/or a wireless communication interface for wireless connection to a computer network. For example, a cellular connection, a Wi-Fi connection, a Bluetooth connection, and other connections may be present in the computer. Such connections support communication with other devices, such as to support voice or data communications.

The computer 500 may have various input device(s) 514 such as a various pointer (whether single pointer or multi-pointer) devices, such as a mouse, tablet and pen, touchpad and other touch-based input devices, stylus, image input devices, such as still and motion cameras, audio input devices, such as a microphone. The computer may have various output device(s) 516 such as a display, speakers, printers, and so on, also may be included. These devices are well known in the art and need not be discussed at length here.

The various storage 510, communication connections 512, output devices 516 and input devices 514 can be integrated within a housing of the computer, or can be connected through various input/output interface devices on the computer, in which case the reference numbers 510, 512, 514 and 516 can indicate either the interface for connection to a device or the device itself as the case may be. An operating system of the computer typically includes computer programs, commonly called drivers, which manage access to the various storage 510, communication connections 512, output devices 516 and input devices 514. Such access generally includes managing inputs from and outputs to these devices. In the case of communication connections, the operating system also may include one or more computer programs for implementing communication protocols used to communicate information between computers and devices through the communication connections 512.

Any of the foregoing aspects may be embodied as a computer system, as any individual component of such a computer system, as a process performed by such a computer system or any individual component of such a computer system, or as an article of manufacture including computer storage in which computer program code is stored and which, when processed by the processing system(s) of one or more computers, configures the processing system(s) of the one or more computers to provide such a computer system or individual component of such a computer system. Each component (which also may be called a “module” or “engine” or “computational model” or the like), of a computer system such as described herein, and which operates on one or more computers, can be implemented as computer program code processed by the processing system(s) of one or more computers. Computer program code includes computer-executable instructions and/or computer-interpreted instructions, such as program modules, which instructions are processed by a processing system of a computer. Generally, such instructions define routines, programs, objects, components, data structures, and so on, that, when processed by a processing system, instruct the processing system to perform operations on data or configure the processor or computer to implement various components or data structures in computer storage. A data structure is defined in a computer program and specifies how data is organized in computer storage, such as in a memory device or a storage device, so that the data can accessed, manipulated, and stored by a processing system of a computer.

Disclosed below is an example of reduction to practice of the methods of the disclosure. In this example, the nanoparticle corona phase (CP) offers a unique materials design space for constructs capable of molecular recognition (MR) for sensing applications. Single-walled carbon nanotube (SWCNT) CPs have the additional ability to transduce MR through its band gap photoluminescence (PL). DNA oligonucleotides are well-known to disperse SWCNTs through forming CPs and can be manufactured with molecular precision. Nevertheless, no generalized scheme exists for the de novo prediction of SWCNT MR based on these CPs due to their sequence-dependent three-dimensional complexity. This example generated a DNA-SWCNT PL response library and leverages machine learning (ML) techniques to understand the DNA sequence dependence of MR. Both local features (LFs) and high-level features (HLFs) of the DNA sequences were utilized as model inputs. In this example, the DNA sequences used to generate the response library were DNA strands of length between 12 and 40 nucleotide bases (NBs), and 176 randomly selected DNA sequences were selected to generate the response library. Out-of-sample analysis of the ML model showed significant correlations between model predictions and actual sensor responses for 6 out of 8 experimental conditions. Different HLF combinations were found to be correlated with sensor responses for each analyte, offering mechanistically differentiable design parameters for these systems. Furthermore, models utilizing both LFs and HLFs show improvement over that with HLFs alone, demonstrating that DNASWCNT CP engineering is more complex than simply specifying molecular properties. An ML-guided approach can be used for nanoparticle CP engineering with relatively few experiments within a high-dimensional design space.

As such, the following example demonstrates that a machine learning approach can be used to develop SWCNT-based corona phase molecular recognition (CoPhMoRe) sensors. These SWCNT CoPhMoRe sensors can function effectively as artificial antibodies that can detect more classes of molecular targets, be more stable in different environmental conditions, and be manufactured with relative ease compared to their biological counterparts. A long-standing challenge in the field is the development of a non-heuristic system for CoPhMoRe design. This problem has a high-dimensional design space. The disclosure involves a highly standardized and large dataset of CoPhMoRe for sensor applications in the context of developing sensors for aquaculture monitoring. The algorithm modeled both low level and high-level aspects of the corona phase design and resulted in predictive models with relatively few experiments considering the size of the design space. CP features associated with MR against analytes of interest are also discovered and disclosed. This example can serve as the basis for development of other CoPhMoRe sensors against other targets of interest.

### Example: DNA Single-Walled Carbon Nanotubes

### Introduction

Antibodies are the most well-known nano-scale constructs capable of molecular recognition (MR) through adsorption to an intended target. Their generation and implementation since 1975 have enabled paradigm shifts in the biomedical sciences. As the MR component of a large proportion of rapid tests and laboratory assays, they are integral in chemical detection, food safety, and physiological sensing. One of their most recognizable applications is the home pregnancy test, where monoclonal antibodies against the human chorionic gonadotropin is used in a lateral flow assay. As targeted therapeutics, or biologics, they are a widely produced class of pharmaceuticals that enable precision treatment of cancer and autoimmune conditions. However, despite extensive use, their design still involves a selection process based on biological machinery. Recently, work has also explored synthetic MR design, including nucleotide aptamers, non-immunoglobulin protein scaffolds, and molecularly imprinted polymers. Potential limitations of these alternative approaches include high cost, low stability, inability to detect different classes of analytes, and most importantly, the lack of a data driven method for design that is able to build upon past experimental results.

An emerging area of synthetic MR that involves the design of the nanomaterial corona phases (CP) is corona phase molecular recognition (CoPhMoRe). The CP of a nanoparticle is the thermodynamically controlled coverage of a material's surface formed from adsorbed molecules. These non-covalent modifications, whether from synthesis or the environment, often serve as the interface that determines a material's properties. In the case of single-walled carbon nanotubes (SWCNTs), their aqueous dispersion through adsorption of small molecules or polymers form surface CPs that can be capable of MR. Furthermore, the discovery that binding events can be transduced through the SWCNT's intrinsic band-gap photoluminescence (PL) led to a series of studies that demonstrated promising photophysical detection of multiple classes of analytes, including reactive species, metal ions, small molecules, and biological macromolecules. One advantage of such a system is that the SWCNT material functions as both MR binder and optical signal transduction sensor, which minimizes interfacial losses that may occur with other two-component designs.

SWCNT CoPhMoRe generation also faces a similar challenge of an enormous design space, starting with the need to formulate of a large library of unique CPs that can also stably disperse single SWCNTs in the solution phase. Fortunately, single-stranded DNA molecules have been demonstrated to stably disperse SWCNTs and also are a common class of polymers that can be synthesized rapidly with molecular precision. Thus, DNA-SWCNTs have been a rich resource for MR design. The nucleotide base (NB) dependent nature of interactions between DNA and SWCNTs have been studied both computationally as well as experimentally at the single-molecule and short motif level. However, there currently exists no intuition to effectively design DNA CPs for the purposes of MR. The most common approach is a systematic enumeration of the sequence design space guided by global intuitions on sequence composition. Considering that there are 4^{L} permutations of DNA sequences (L being the sequence length), which then encodes for complex secondary and tertiary DNA structures that are also influenced by unique interactions from adsorption to the low-dimensional SWCNT, intuitive or random screening-based search methods are highly inefficient for converging on promising targets.

Recently, machine learning (ML) techniques have been of considerable interest in exploring materials design spaces. The goal of these methods is to perform classification and prediction tasks to optimize predefined metrics related to materials properties. Previous efforts have involved machine learning-based example of DNA-SWCNT, specifically for the application of sequence dependent SWCNT chirality separation in aqueous two-phase systems. By limiting the DNA strand length to 12 NBs, 82 strands were modeled using a panel of learning algorithms to demonstrate a higher than 50% success rate of finding chirality separation sequences. However, to date, no such computational example or uniformly controlled large dataset exists for the evaluation of DNA-SWCNT CoPhMoRe development.

This example demonstrates a first-of-its-kind approach using machine learning (ML) to identify and inform DNA-SWCNT MR. Specifically, the goal is to use ML to predict which DNA sequences enable better SWCNT PL analyte responses for each specific experimental condition. A panel of analytes (cadmium, enrofloxacin, chloramphenicol and semicarbazide) were selected based on the need for rapid and quantitative testing technology for adulteration in the aquaculture supply chain. To enable this ML approach, DNA-SWCNT PL spectra change from analyte exposure were collected to create the largest sensing response library to date for 176 randomly chosen DNA sequences. From this training data, ML predictions for DNA sensor responses were made using the following three steps. First, a convolutional neural network (CNN) was used to predict shorter-length DNA motifs correlated with photophysical responses, which is referred to as local structure predictions. Second, independent features were created based on a principal components analysis (PCA) vectorization of 40 high-level features (HLFs) (e.g., molecular weight, melting point, dimers, etc.). Third, these HLF and CNN model outputs were then both used as independent features for gradient-boosted decision trees (GBDTs) to produce the final predictions regarding whether DNA sequences can produce promising sensor candidates for each analyte and sensing environment. The example demonstrates that these ML models can significantly predict DNA-SWCNT MR with relatively few data points. Through interpretation of the HLFs that were significantly correlated with improved MR, general properties that increase sensor response can also be identified (e.g., decreasing melting temperatures, increasing adenine content, and decreasing thymine content). As a whole, it is shown that DNA-SWCNT sensors offer unique NB dependent MR that is capable of differentiating analytes and measurement conditions. While still a computationally and experimentally challenging problem, this example offers the first systematic insights into DNA sequences effects and experimental design considerations for future computationally driven CoPhMoRe studies.

### Data Collection and Processing

Example Organization and Data Collection. Many primary food supply chains (FSCs) are often a source of serious quality and safety problems. These issues can arise from substandard practices and poor operational conditions, but also intentional or economically motivated adulteration. Some of the specific agents presenting threats to human health in the aquaculture FSC include heavy metal contamination of water sources from industrial mining and antibiotic adulteration above acceptable levels. Since FSCs are typically complex and change dynamically over time, part of determining the appropriate regulatory actions and ensuring consumer safety involves developing rapid testing capabilities to detect adulterants of interest. This example, based on a survey of aquaculture markets, focuses on developing sensor elements against cadmium ions and three small molecule antibiotic species: enrofloxacin, chloramphenicol, and nitrofuran's degradation product, semicarbazide. An analyte concentration of 100 μM was chosen based on previously measured k_{d} values of SWCNT CoPhMoRes. Additionally, a dataset containing previous results of DNA-SWCNT sensors against arsenite (AS³⁺) and arsenate (AS⁵⁺), two other candidates of interest, were included in the computational analysis as comparison.

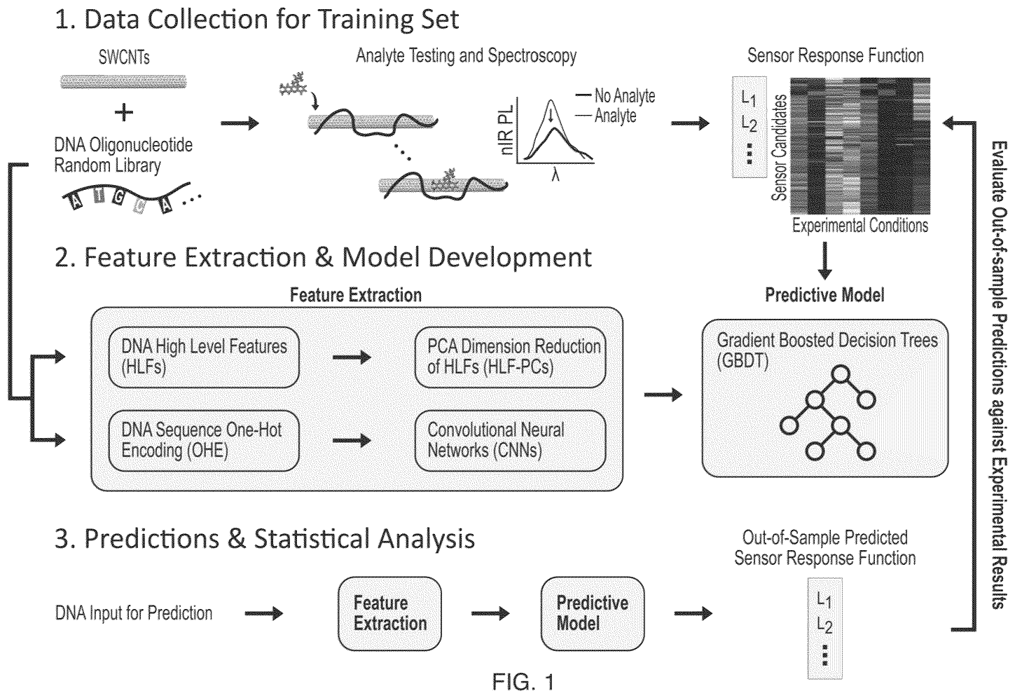

An overview of the methodological approach is shown in FIG. 1. This example revolves around computationally exampling the DNA sequence dependence of DNA-SWCNT photophysical sensor constructs against the analytes of interest. SWCNTs synthesized from the high-pressure carbon monoxide (HiPco) method were used, which contain a range of different chirality small diameter SWCNT species that can be concurrently probed. Each sensor construct was comprised of a colloidal aqueous dispersion of SWCNT using a single unique sequence of randomly generated single-stranded DNA, with the design space constrained to all DNA strands of length between 12 and 40 NBs. Shorter DNA lengths were not considered due to poor dispersion stability, potentially resulting in aggregation after analyte addition. Longer DNA lengths were not considered due to increased CP stability with polymer length. Since the goal was to engineer DNA-SWCNT constructs to interact with analytes from physical adsorption and subsequent CP modulation, a CP that is too stable may be less likely to result in adsorption and signal transduction. However, despite using the limited sequence range, the design space is still considered innumerable from an experimental point of view at greater than 1×10²⁴ permutations just for DNA molecules of length 40.

Referring now to FIG. 1, there is shown an overall experimental and computational scheme for data generation, including the collection, processing, modeling, and analysis of the photophysical response data from sensors. 1) First, DNA-SWCNT dispersions were prepared from a library of sequences. These candidate MR binders were then tested against the analytes of interest under different pH conditions, resulting in nIR spectral changes measured between 850-1250 nm. The model input is created by converting the before and after spectra from each analyte incubation into an optimizable score (see Equations 1 and 2 herein) through the sensor response function. 2) The DNA sequences were encoded via 2 methods: direct vectorization/one-hot encoding or through calculations of common high-level features (HLFs). Using these two types of inputs, predictive models using gradient boosted decision trees (GBDTs) were created. 3) Finally, the model was used to score potential, candidate MR binder designs which were evaluated in out-of-sample analyses against laboratory results.

Each chosen DNA sequence for the candidate MR binders was used to disperse SWCNT in aqueous solution via tip ultrasonication and purified through centrifugation using methods previously described. UV-vis-nIR absorption spectroscopy was used to assess dispersion quality and concentration prior to sensing studies. The experiment itself was performed using a custom high throughput nIR spectroscopy setup, exciting samples comprised of either control or analyte at chosen experimental conditions with a 785 nm laser, and measuring PL in the range of 950-1250 nm.

Recent studies showed that SWCNT PL and analyte responses can be strongly dependent on solution and experimental conditions. To achieve the best controlled results and to minimize experimental variation from method error, the following standard experiment was developed:
The DNA corona phase is highly dependent on solution pH, and likely adopts two different equilibrium conformations. Specifically, pH 6 and 8 conditions were chosen for each DNASWCNT as two independent sensing states.
The test solution was buffered to 0.1 M ionic strength in sodium phosphate and allowed to equilibrate for a minimum of 6 hrs against known dilution effects.
It has recently been found that DNA-SWCNT PL quantum yield, defined as photons emitted per particle, decreases with increasing excitation fluence. To mitigate this effect maximally without significantly increasing experimental time, excitation fluence at the sample was limited to and controlled at 1.67 mW/μm² for all experiments.
DNA-SWCNT is also known to associate to form loosely structured aggregates in solution. To mitigate these effects, SWCNTs were diluted to 0.5 mg/L, lower than previous studies, and were continuously agitated during analyte incubation.
SWCNT PL responses can also have kinetics on the order of hours, especially in the case involving DNA and metal ions. Thus, measurements were performed after exactly 1-hour incubation to assure both reproducibility and sufficient measured response.

A total of 176 unique sequences were chosen as CP sensor candidates to test against 8 experimental conditions (2 pH conditions and 4 analyte conditions). One portion of the library was chosen randomly with respect to each NB, where each base choice was drawn with 1/4 probability. Another portion of the library was constructed from random pairs of NBs (e.g., AATTGGCC . . . ). This bias was chosen due to the larger relative size of the SWCNT diameter to an individual NB, making two consecutive NBs more likely to modulate physical effects. To apply the newly proposed methods to an existing dataset in literature, sensor screening results against As³⁺ and As⁵⁺ was extracted from previous work, having 22 and 14 unique non-random DNA sequences, respectively.

Data Processing and Model Inputs. In the PL experiments, a sensor response was defined as the change in PL emission spectra between a control condition and one incubated with analyte. Since the HiPco SWCNT sample contained a distribution of semiconducting SWCNT chiralities, the nIR emission spectra is a linear combination of individual chirality emission peaks. Given that each SWCNT chirality species' emission peak can change in intensity and/or wavelength, and/or broaden after analyte interactions, it was decided not to fit, or deconvolute, the spectra during analysis due to the number of fitting variables.

To convert optical spectral responses into optimizable numerical values (i.e., the dependent variable for the ML models), two types of observably modified spectral features after analyte interaction were defined: PL intensity and PL wavelength. To capture intensity modulations, an integrated normalized intensity change between the experimental and control was calculated. To capture the trend in wavelength modulations or peak shifts, a term describing the overall shape change of given a similar intensity change was calculated. The shape term is designed to be smaller when either one of the following conditions hold, based on a manual review of the typical variation between DNA-SWCNT sensor responses:
The sensor response curve intensity is shifted up or down proportionally at all points (i.e., c(λ)=b*f(λ), ∀λ, where b∈ [symbol] )
The highest peak of the sensor response curve, and the rest of the curve, are each shifted up or down proportionately at all points, but by different amounts (i.e., c(λ*)=b₁*f(λ*) for λ*=arg max_{λ}(c(λ)=b₂*f(λ), ∀λ≠λ*, where b₂, b₂∈ [symbol] )

The combined sensor response function is defined as:

[symbol]

$$Sensor Response ≡ \frac{{\int }_{a}^{b} \vert f ( λ ) - c ( λ ) \vert d λ}{{\int }_{a}^{b} c ( λ ) d λ} + α {\int }_{a}^{b} \vert g ( λ ) - β \vert d λ \qquad ( 1 )$$

$$g ( λ ) ≡ ( \frac{f ( λ )}{{f}_{max}} - \frac{c ( λ )}{{c}_{max}} ) \qquad ( 2 )$$

$$β ≡ \frac{1}{a - b} {\int }_{a}^{b} \frac{f ( λ )}{{f}_{max}} - \frac{c ( λ )}{{c}_{max}} d λ \qquad ( 3 )$$

The shape term accomplishes this by summing at each wavelength the difference in proportion between intensity for the wavelength and the highest peak of the analyte and response curves. This is accomplished with the g(λ) term. The subtraction of the β term ensures that when the highest peak of the curve is the same intensity, but the rest of the curve is shifted down proportionately at all points (i.e., c(λ*)=f(λ*) for λ*=arg max_{λ}(c(λ)), but c(λ)=b*f(λ), ∀λ≠λ*, where b∈real numbers set (R)), that β=g(λ), ∀λ≠λ*. Where the data wavelength range is between a and b, c(λ) is the PL spectra of the control DNA-SWCNT at wavelength λ, cₘₐₓ is the maximum PL spectra of the control across all wavelengths, f(λ) is the PL spectra of the experiment sample at wavelength λ, fₘₐₓ is the maximum PL spectra of the experiment sample across all wavelengths, and the linear proportionality constant between the two sides of the sensor response function. The shape term is comprised of g(λ) and 3, where g(λ) represents the proportional difference in intensity at wavelength λ compared to the highest peak, between the analyte and response curves, and where subtraction of the term ensures a small shape term for the motivating examples described earlier in the section.

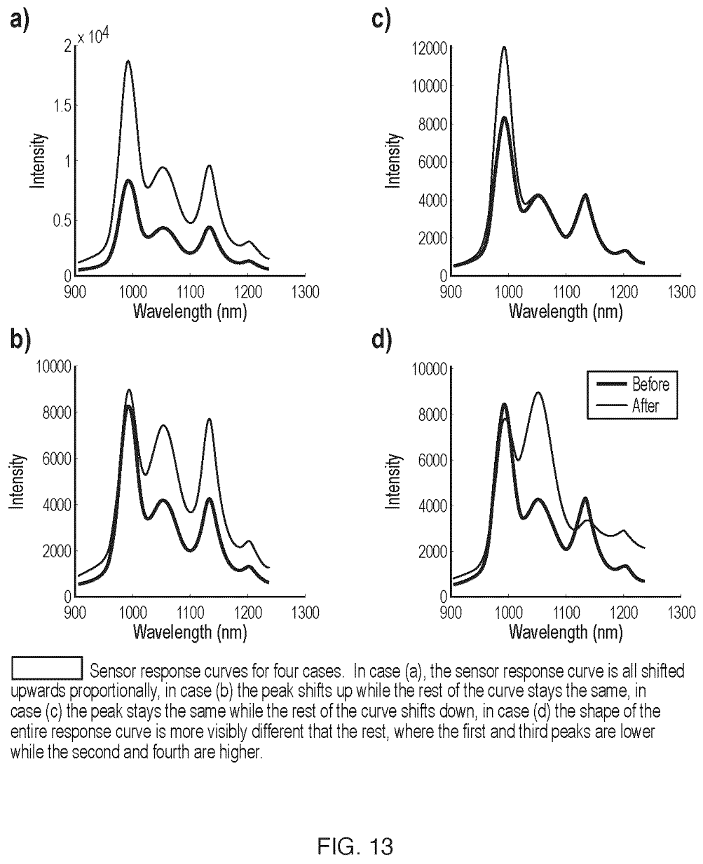

The PL data from this example was collected between 850 nm and 1250 nm. Since analyte interactions from MR can result in both PL intensity and/or wavelength changes as a function of experimental conditions, an intuitive decision was made to assign both sides of the sensor response function with equal weight as to maximally capture any optimizable features. The value of α=0.0113 was empirically determined by matching the ranges of measured values of the intensity and wavelength sensor response terms for the whole experimental dataset. (For more motivation of the sensor response function, including examples of sensor response curves, see FIG. 13)

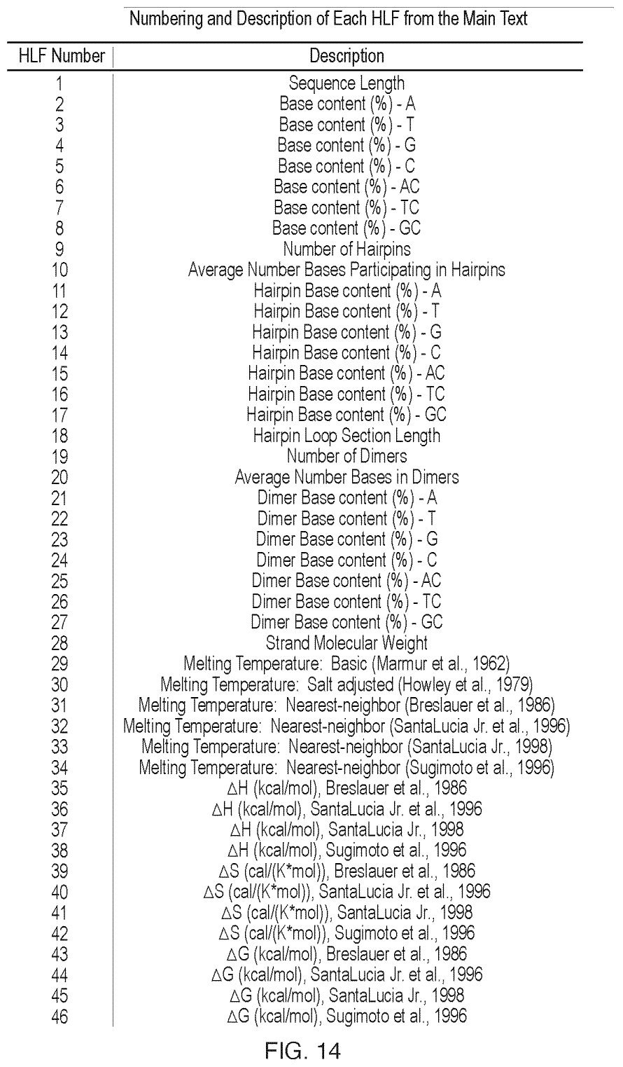

To create the covariates (i.e., independent features) for the ML predictions, HLFs were calculated for all DNA sequences. These were biophysical metrics used to evaluate a single-stranded DNA sequence. Some HLFs were directly calculated from the sequence primary structure, including strand lengths and percentages of each base type. Derived HLFs required existing models of DNA structure. It was assumed 25° C. and 0.1 M salt concentration when calculating non-covalent DNA interactions such as strand hybridization and hairpin formation. Lastly, thermodynamic properties were also predicted for the solution phase single-stranded DNA strands using well known models for ΔS, ΔH, ΔG, and melting temperature. While it is difficult to know the exact influence of these derived features for DNA due to the close proximity of the strand to the unique hydrophobic and curved surface of the SWCNT, this prior knowledge likely biases DNA CP structure and behavior. A description of each of the 46 HLFs is detailed at FIG. 14.

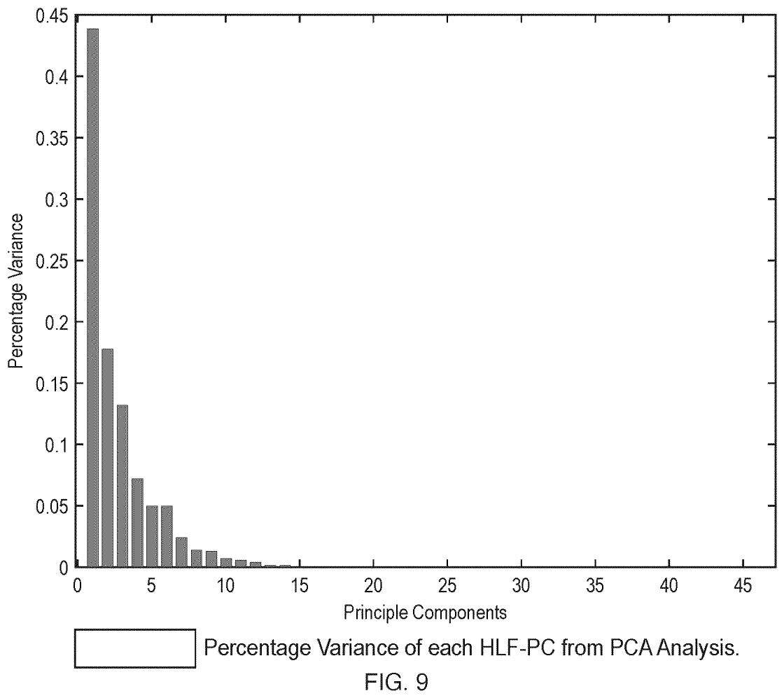

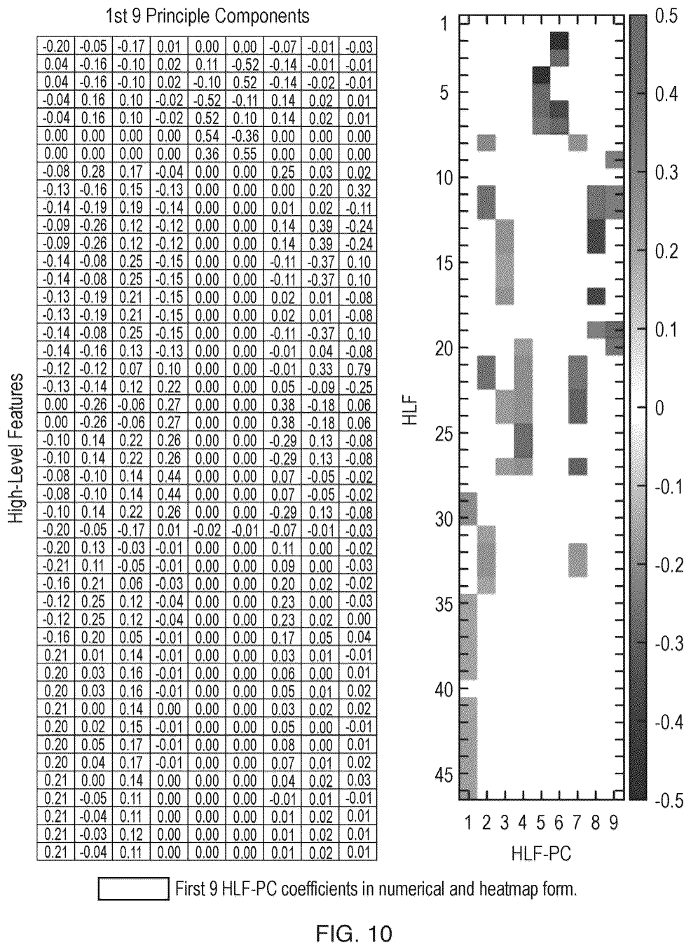

Finally, the multiple HLFs defined in the form above were correlated with similar physical properties and thus are interdependent (for example increased sequence increases melting temperature). To specify independent parameters for model input, PCA was performed on the 46 HLFs for one million randomly generated sequences of lengths 12-40 NBs. The resulting PCA coefficients were used to convert HLFs associated with each sequence into a lower-dimensional space of model inputs that were referred to as HLF principal components (HLF-PCs). Of note, the first 9 principal components were able to capture over 95 percent of the total HLF variation. (See FIGS. 9 and 10 for HLF-PC variance and coefficients.)

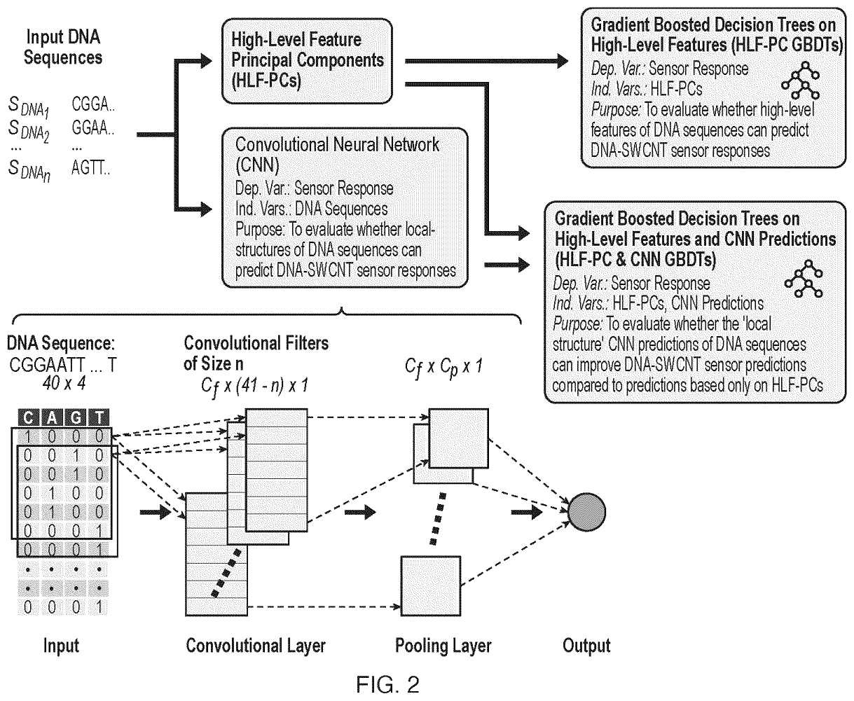

Model Specification and Training. The machine learning analysis relied on two-stage modeling for each experimental condition, summarized in FIG. 2. The rest of the section describes model specification, hyperparameters, and training in more detail.

CNNs were used to evaluate the effect of sequence dependent DNA local structure on optical responses. CNNs excel at certain tasks (e.g., image recognition) by identifying local structures in inputs (e.g., curves within images) and have successfully previously been utilized to predict transcription factor binding DNA motifs. For this example, it was hypothesized that CNNs can identify DNA motifs responsible for binding sites against the target analyte, identifying features correlated with stronger optical responses.

The CNN architecture is detailed in FIG. 2. CNNs were fit using the sensor response in Equation 1 as the dependent feature, one-hot encoding (OHE) of the DNA NBs as independent features, and implemented with Tensorflow. OHE refers to the direct vectorization of the DNA sequence into n×4 matrices where n is the sequence length. Various architectures of single-layer CNNs were considered, including number of convolutional filters c_{f}∈{2, 4, 8, 16, 32, 64}, and motif sizes n∈{4, 6, 8} (filter size for pooling layers is set proportional to motif size). Multiple regularization hyperparameters were considered, including dropout d∈{0, 0.5} and number of training epochs nₑ∈{100, 150, 200, 250, 300, 350, 400, 450, 500}. Model hyperparameters were selected out-of-sample, using Bayesian optimization implemented with GPyOpt. The CNNs architecture and independent feature encoding are displayed in FIG. 2. Out-of-sample CNN predictions were made for each experimental condition (analyte/pH) and DNA sequence, and used as an input to GBDTs. The disclosure provides detailed information about how the CNNs were trained, hyperparameters were selected, and out-of-sample predictions were made.

Final predictions on DNA-SWCNT outcomes were made with GBDTs implemented with XGBoost. GBDTs are ensemble models that fit decision trees stage-wise to minimize residual error, and were selected for their strong performance in data science competitions and their ability to learn complex interactions given limited data. GBDTs were fit with the sensor response as the dependent feature, and HLF-PCs and local structure CNN predictions as independent features. Hyperparameters to control model complexity and regularization included tree depth t_{d}∈{3, 4}, learning rate rho∈{0.01, 0.025, 0.05, 0.1}, and number of trees tₙ∈{100, 250, 500}. The disclosure provides detailed information about how the GBDTs were trained, hyperparameters were selected, and out-of-sample predictions were made.

Evaluating Predictive Power. Out-of-sample predictions were evaluated for each experimental condition. Specifically, Pearson correlations of predicted and actual sensor responses were calculated for GBDTs trained with HLF-PCs as independent features. Corresponding p-values were used to assess whether HLF-PCs can predict DNA-SWCNT outcomes. The analysis was repeated for GBDTs trained with both HLF-PCs and local structure CNN predictions as independent features. A p-value was calculated for the difference of these two correlated Pearson correlation coefficients, to assess model improvement from the addition of CNNs.

Evaluating Significant Features. To determine significant features for the HLF-PCs, linear regression models were fit for each experimental condition, using HLF-PCs as independent features and sensor response as the dependent feature. Linear regressions were selected due to their interpretable p-values for each fitted model coefficient. This allows the HLF-PCs to be qualitatively interpreted in order to understand how HLFs impact DNA-SWCNT outcomes.

Assessing Number of Samples for Predictive Power. To assess the predictive power of the GBDTs as a function of the number of samples, the following was done. For each experimental condition where predictive power was previously established, and for any number of samples n∈{11, 16, 21, . . . , 176}, n DNA-SWCNT samples were randomly chosen and GBDTs were retrained to produce out-of-sample results for each sequence using the same methodology as before. The Pearson correlation coefficient was then calculated for the predicted versus actual sensor response. This process was repeated for 100 random samples for each value of n<=36, and 25 random samples for all values of n>=41 (to account for there being more variation in the Pearson correlation coefficient for smaller sample sizes).

### Results and Discussion

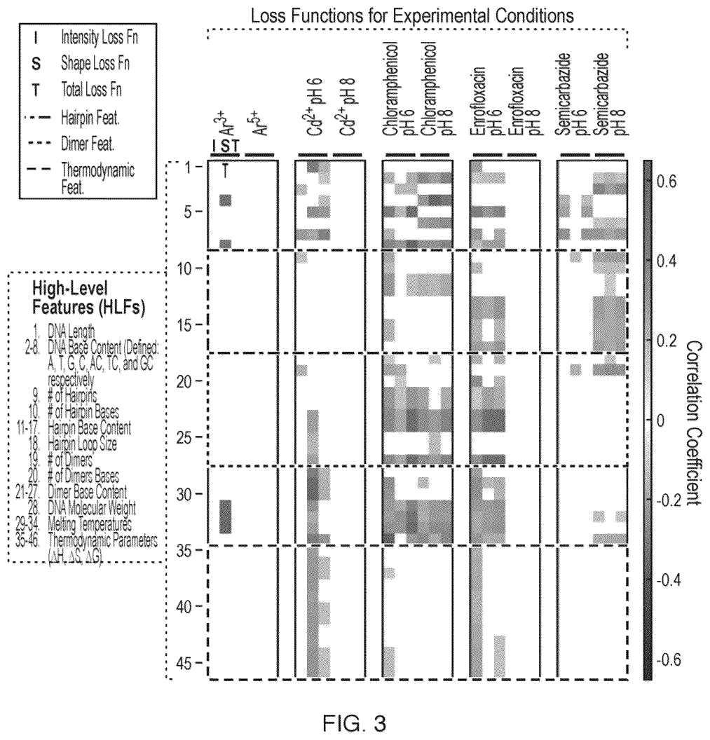

Experimental Output Interpretation. One representation of the entire dataset is shown at FIG. 3. Here, HLFs of each sequence were first calculated. The Pearson correlation coefficient between HLFs and sensor response is graphically shown for the cases where the p-value <0.05.

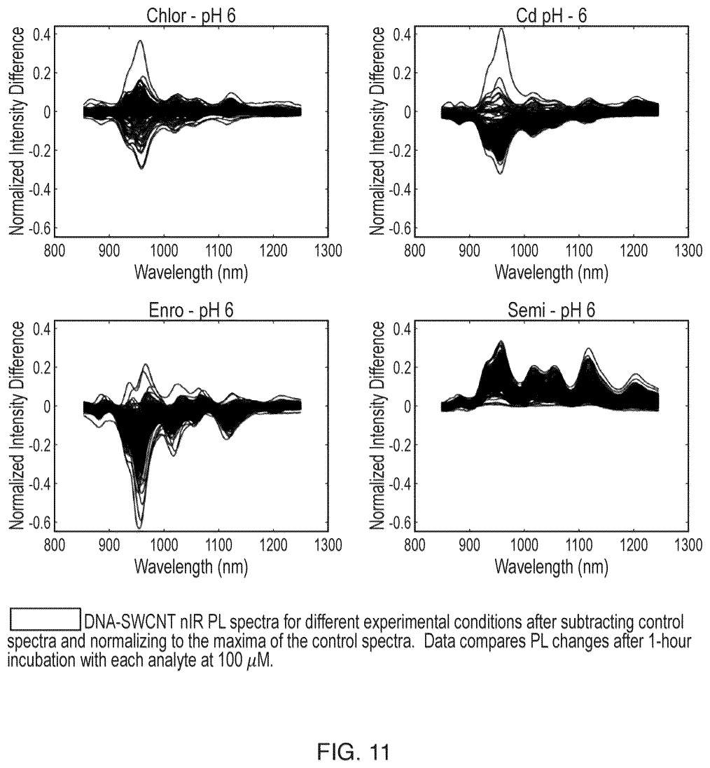

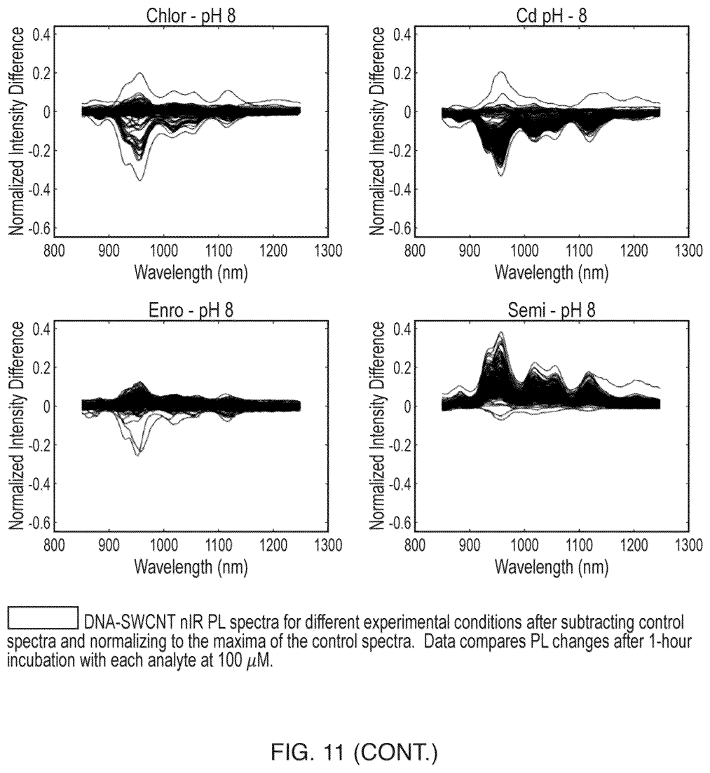

The following general observations can be made regarding the experimental results:
The pH 6 and 8 conditions, while using the same DNA CP, have different photophysical response correlations with the HLF panel and likely different surface structure interactions. Thus, these pH conditions can be treated as separately optimizable MR conditions.
Decreased DNA length, increased adenine content, and decreased cytosine content were positively correlated with improved MR responses. These characteristics are positively correlated with more responsive and less stable DNA CPs, supporting the choice of limiting the example to shorter DNA strands.
While the intensity and shape sensor responses were generally congruent, they differed sufficiently and thus were necessary to provide orthogonal information to the algorithm.
The correlation with DNA secondary structure implies that strand-strand interactions play a major role in organizing the SWCNT CP.
Each experimental condition and analyte response appeared to have a unique HLF correlation “barcode,” generally suggesting that the MR is mechanistically different and can be differentiable between the individual cases. Changes in PL emission spectra associated with these experimental conditions were also observed to be uniquely different (see FIG. 11). This observed variation based on analyte composition and CP structure highlights the depth and utility of a CoPhMoRe system for engineering specific interactions.

Generally, if a HLF associated with a DNA CP is correlated to sensor responses for an experimental condition given a sufficient number of data points, then such sensors were likely DNA sequence optimizable. However, the lack of correlation does not necessarily indicate poor sensing because there can exist photophysical PL modulating mechanisms that are DNA sequence independent and intrinsic to that of the SWCNT alone (for example, analytes that react directly with the SWCNT chemically while bypassing any selectivity imposed by the CP). While less can be concluded about the arsenic data due to the significantly lower number of data points and the intuitive manner from which they were chosen, the observed correlation of sensor response with guanine and length were consistent with the conclusions of a previous example.

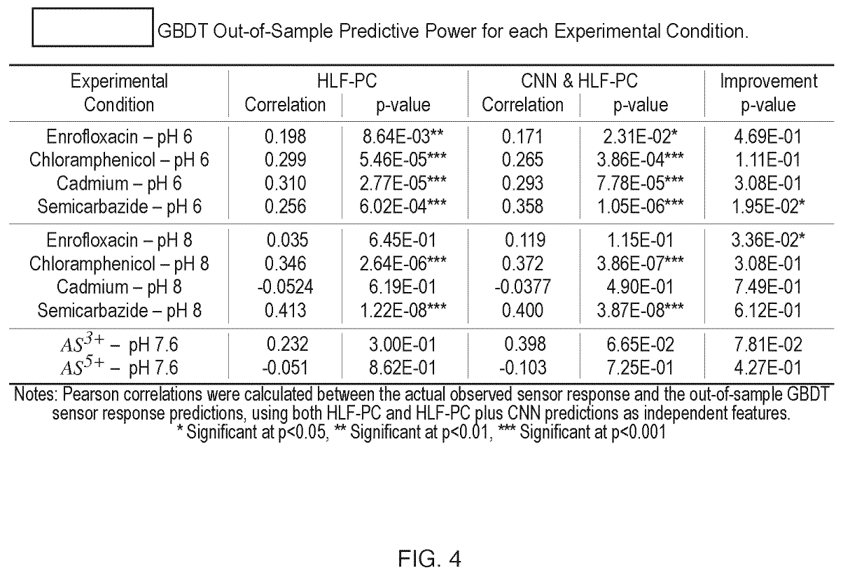

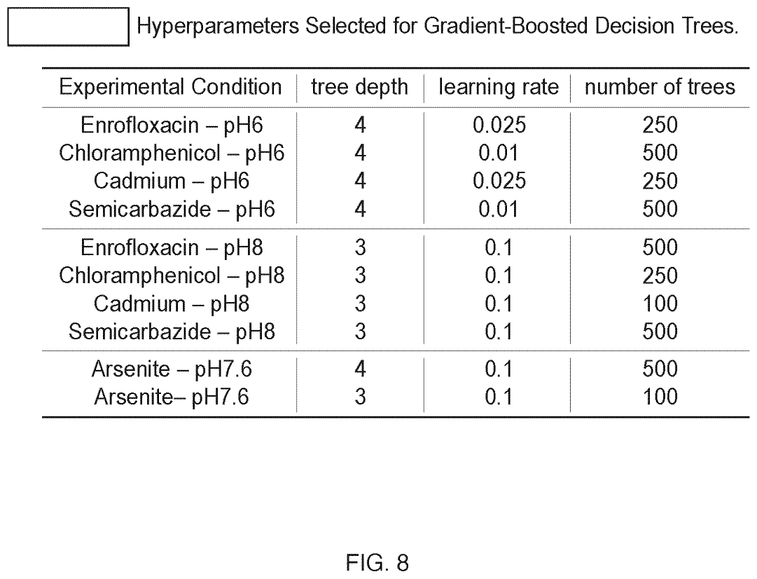

Predictive Power. For each experimental condition, GBDTs were trained and used to predict the out-of-sample sensor response for each DNA sequence (see FIG. 8 for model hyperparameters). FIG. 4 displays Pearson correlations between the predicted and actual sensor responses, and their corresponding p-values. This was done with and without the local structure CNN predictions as model inputs.

GBDTs achieved statistically significant predictions for six of eight experimental conditions in the example, both with and without the local structure predictions from the CNN as model inputs. Predictions were significant for all analytes at pH 6, and for chloramphenicol and semicarbazide at pH 8. Pearson correlation coefficients were as high as 0.413 for semicarbazide at pH 8, indicating that 17.1% of the variation in the sensor response can be explained by the predictive model. The corresponding p-value of p=1:22×10⁻⁸ is convincing for the first CP sensor of this kind. Furthermore, evidence was found that local structure CNN predictions can improve performance, and their inclusion as a model input improved correlations with statistical significance for two experimental conditions at the p<0.05 significance level (semicarbazide pH 6 and enrofloxacin pH 8). For the previously published arsenic data, with a smaller sample size, AS³⁺ achieved significant predictions and model improvement at the p<0.1 significance level.

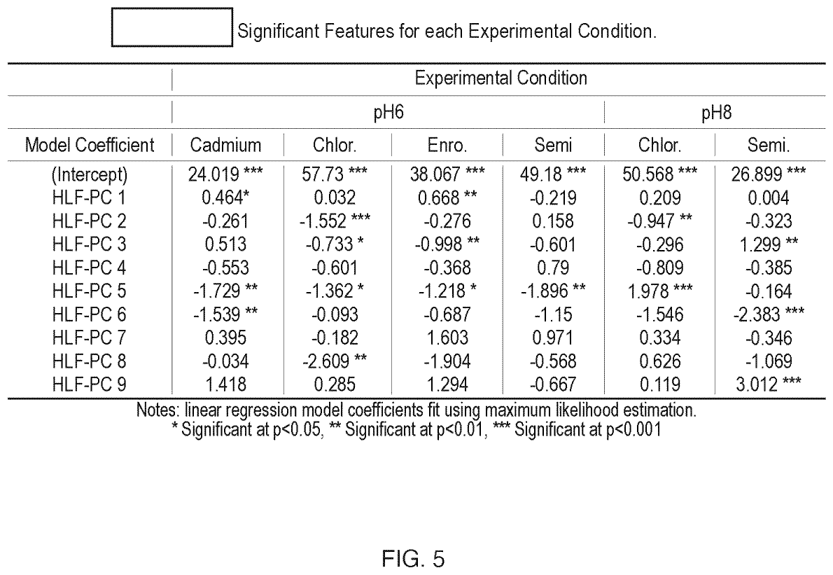

Evaluating Significant Features. To evaluate significant features, a linear regression was fit for each experimental condition with HLF-PCs as independent features and the sensor response as the dependent feature. This was done for all six experimental conditions from FIG. 4 with significant predictions at p<0.05. Fitted model coefficients and statistical significance are shown in FIG. 5.

Interpretation and Overall Observations. To interpret the significance of HLF-PCs, the HLFs with higher magnitude coefficients for each PC were examined (See FIG. 10 for graphical presentation). Each HLF-PC and some of their related physical properties are listed below (arrows represent the direction of correlation with the HLF-PC, letters show the composition of individual or pairs of NBs, brackets ‘( )’ group base compositions that are modulated together):
HLF-PC 1: decreased melting temperature with increased ΔS, ΔH, and ΔG
HLF-PC 2: increased melting temperature with increased (GC) and decreased (AT) in secondary structures
HLF-PC 3: increased (GC) in secondary structures
HLF-PC 5: decreased G and increased (C, AC, TC)
HLF-PC 6: decreased (A, AC) and increased (T, TC)
HLF-PC 8: increased (A,T) and decreased (G, C, GC) in hairpins. Increased number of dimers
HLF-PC 9: Increased number of hairpins and dimers, decreased A, T in them, decreased number of NB in each dimer

For the set of experimental conditions, HLF-PC 2 and 6 were both negatively correlated to sensor responses. General properties correlated to sensor responses have been learned, such as: decreased melting temperatures, increased adenine content, and decreased thymine content. HLF-PC 5 had the largest change in sensing between the two different pH conditions, with strongly negative correlations in pH 6 conditions, and a strongly positive correlation for chloramphenicol at pH 8. Structurally, sensing of semicarbazide at pH 8 prefers increased number of likely secondary structures while chloramphenicol at pH 6 is the opposite.

As a whole, these significant correlations with HLF-PCs can be used to improve the detection level of sensors while performing fewer experimental iterations as compared to a random search. For example, the detection of semicarbazide at pH 8 is correlated with increased HLF-PC 3, decreased HLF-PC 6, and increased HLF-PC 9. Thus, subsequent sequence search libraries should bias towards increased (GC) in secondary structures, increased (A, AC), decreased (T, TC), increased number of hairpins and dimers, decreased A, T in them, and decreased number of NB in each dimer. It is important to note that while these characteristics were significantly correlated to the sensor response, they may explain a small portion of the variance within the known dataset. Thus, guided searches may also include a component of exploration.

The overall differences between the HLF-PC preferences for experimental conditions showed that DNA-SWCNT MR offered unique NB dependent selectivity. It is noted that HLF of DNA molecules play a major role in their SWCNT-adsorbed structure, and thus subsequent analyte interactions. The improvement from the combined HLF-PC and CNN model from that of the HLF-PC alone was the first objective demonstration of the intuitive idea that CP-based MR is more complex than simply specifying a polymer with a set of general properties. However, this improvement was only shown in 2 of the 8 cases. It is possible that a similar improvement can be seen for the rest of the experimental conditions given a large enough sample size.

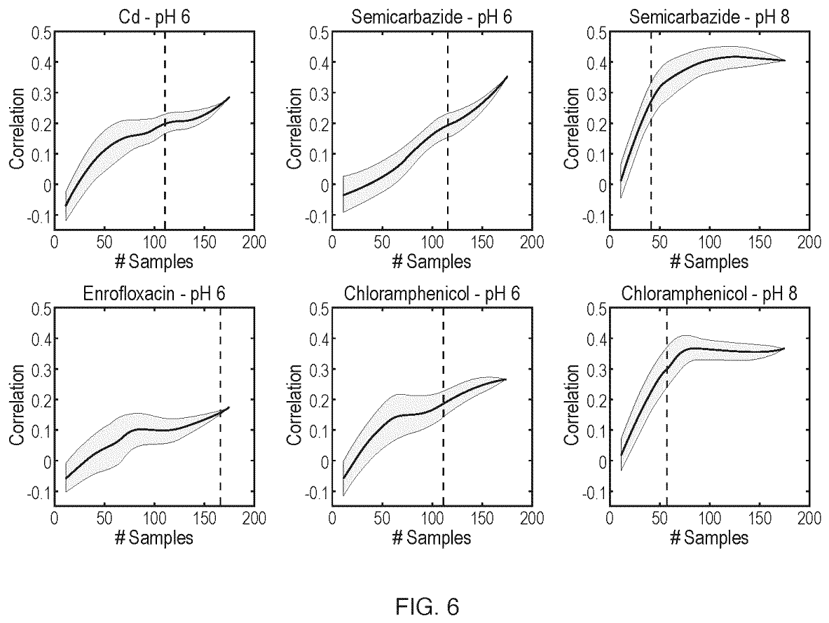

Assessing Number of Samples for Predictive Power. To examine the effect of sample size on out-of-sample correlation for the 6 significant experimental conditions, the average Pearson correlation coefficient between predicted and actual sensor response is plotted versus the number of samples considered for the HLF GBDTs (FIG. 6). From FIG. 6, it was observed that the four experimental conditions at pH 6 all exhibit continued and steady improvement in the out-of-sample predictions as the number of samples increased. In contrast, both experimental conditions at pH 8 achieved higher correlations earlier at around 75-100 samples, but then had decreasing marginal returns in predictive power thereafter.

Generally, the sample size analysis can be used to aid the design of future targeted MR against new analytes using the DNA-SWCNT system. The average number of samples to reach statistical significance was 126 for pH 6 and 49 for pH 8 experimental conditions. Due to the size of the current dataset, the dimensionality of the search space and the nature of extrapolation, it may be difficult to estimate the sample size required for a significant iterative improvement over the current sensor candidates. However, given the few samples required to develop models with significant correlations, the example can be used as an order-of-magnitude estimate for future studies.

### Conclusions and Discussion

The CoPhMoRe design space spans a large range of molecular compositions as well as physical interactions between the analytes, kinetically trapped molecules, and the nanoparticle. While there exists a portfolio of studies on the development of SWCNT-based CoPhMoRe sensors, the complex and often transient mechanisms of interactions between the analyte and the sensor constructs present major challenges for both rational design and optimization of such systems. While the DNA CPs offer a molecularly defined library for sensor discovery, the number of sequence permutations, let alone secondary and tertiary structure from material adsorption, significantly complicates the search and optimization within this design space. Furthermore, the general knowledge gained in recent years regarding DNA and SWCNT interactions has not led to reliable methods of generating sensor elements.

While sensor candidates can be found through systematic or random searches, a machine learning guided method is more adept at solving such a high-dimensional problem. Machine learning techniques are applied in combination with a library of DNA-SWCNTs to evaluate sensor development against analytes of interest in aquaculture. The search was restricted to DNA strand length of 12-40 NBs and the largest CoPhMoRe screen to-date was performed, which included 176 sensors with 8 experimental conditions. Nevertheless, there are 1.21×10²⁴ unique DNA strands of length 40. The results showed that significant predictions can be made for 6 out of the 8 experimental conditions even for the extremely sparse number of samples compared to the dimension of the search space.

The DNA sequences for this example were modeled separately through HLF-PCs or CNNs, incorporating high-level or local structure features respectively. The CNN was constructed through OHE of DNA-NBs, with Bayesian hyperparameter optimization used to find appropriate model architecture and regularization given the amount of available data. HLF-PCs were constructed through PCA vectorization of broadly applied HLFs. Then they were then analyzed together via GBDTs, with out-of-sample predictions showing substantial promise in being able to predict sensor responses.

The fundamental difference between the HLF-PC and the CNN models is one of general known DNA properties versus local sequence features. Interestingly, the combined HLF-PC and CNN model showed improvement from the HLF-PC model in 2 of the cases. This is the first statistical evidence for the importance of local features. While it is likely that many or most DNA-SWCNT responses were dictated by global DNA properties, there may exist an analyte-dependent subset that relies on a more specific sequence-dependent mode of MR.

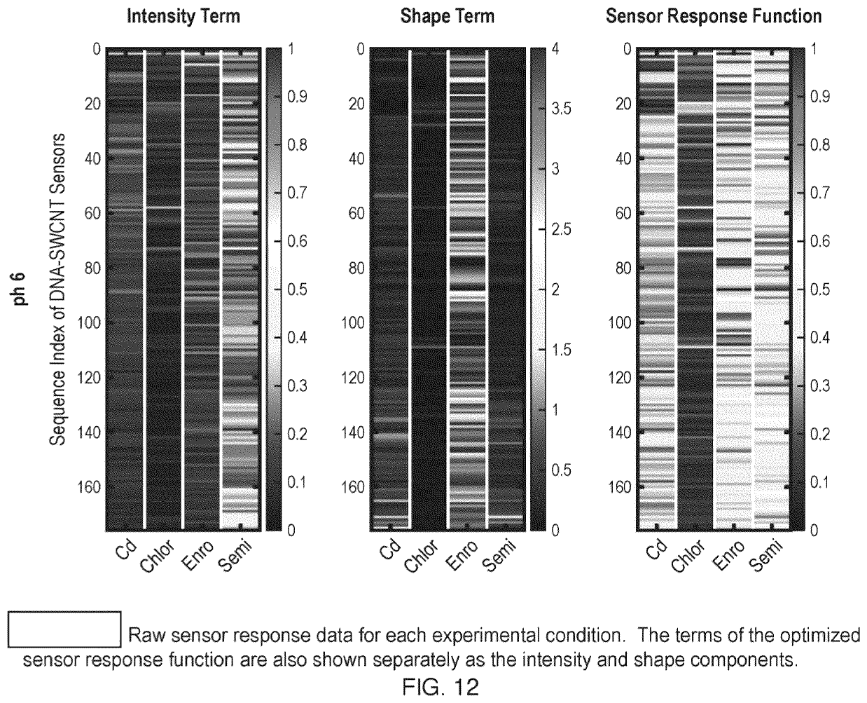

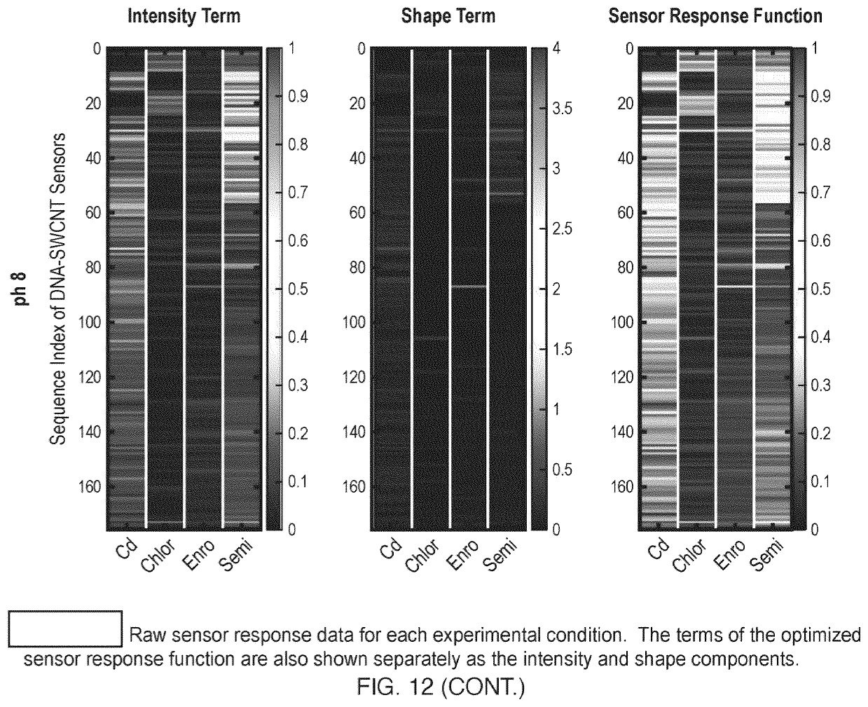

The results also reinforce the idea that each DNA-SWCNT offers multiple independent sensing states as a function of pH. In this example, sensors in pH 6 or pH 8 can be optimized uniquely against each of the analytes through the same sensor response. Through studying HLF-PCs, general properties that improve the photophysical response of DNA-SWCNT include decreased melting temperatures, increased adenine content, and decreased thymine content. Additionally, the raw PL emission changes (see FIG. 12) and the combination of significant HLF-PCs showed that the DNA-SWCNT platform interacted with different analytes in a spectrally and physically differentiable manner. Finally, the results showed that significant predictive models can be created with only about 50-100 samples, providing an improvement for future efforts. It may be recommended that the methodological controls developed here be implemented in future studies to minimize the effect of experimental method error on model predictive power.

From an experimental point of view, the generation and testing of CPs is currently a bottleneck for the execution of ideal studies comprised of thousands of samples. Methods will need to be developed to bypass or automate the process of library SWCNT CP synthesis, sonication, and centrifugation. A second area of potential improvement is the interpretation or vectorization of DNA sequences by considering additional information. Development of these methods will aid in re-scaling of the search space to focus on regions of interest. For example, the well-defined molecular structure of SWCNT should present CP structural biases. CP polymers can self-self-interact either in a collapsed state on the SWCNT surface or after wrapping around. This opens up a new type of length-dependent interaction analysis. In these cases, having an adequate CP structural understanding is paramount. Similarly, additional information regarding DNA tertiary structure with or without a nanomaterial can be incorporated in the vectorization process. Third, while the results inform largely on interpretation of HLFs related to the influence of DNA on CP sensor development, model predicted NB sequences can be leveraged for more granular analysis given that the models are derived from a larger dataset with higher predictive capability. For example, outputs from such models can be used to generate large sequence prediction libraries as input into deep-learning algorithms commonly employed in bioinformatics for the discovery of nucleotide-protein binding motifs.

To conclude, while nanomaterials' unique physical and chemical properties provide promising environments for MR design, the size of the parameter search spaces, and throughput of experiments, are challenges well-suited for ML-based studies. This example demonstrates the feasibility of ML models to analyze relatively few tightly controlled CoPhMoRe sensor studies to provide significant insights into sequence-dependent properties related to sensor-analyte interactions.

### Experimental Methods

Materials. All chemicals were purchased from Sigma-Aldrich (USA) unless stated otherwise. ssDNA sequences were purchased from Integrated DNA Technologies (IDT, USA). HiPCO Raw SWCNTs were used for all experiments and were purchased from Nanointegris (Batch HR27-104).

Preparation and Characterization of SWCNT Dispersions. SWCNT dispersions were prepared by combining 1 mg of SWCNTs and 1 mg of ssDNA in 1 mL of 100 mM NaCl solution. This mixture was tip sonicated (Qsonica Q500 with multi-tip add-on) while cooled by a pre-chilled rack with 0.125 in. probes for 30 min at a power of approximately 22 W (8 tips). Crude SWCNT dispersions were centrifuged two times at 16,000 g for 90 min to remove SWCNT bundles and other solid impurities. The top 80% of supernatant was collected after each round of centrifugation. Absorption spectra of SWCNT dispersions were collected (Cary 5000, Agilent Technologies) to approximate the concentrations of the post-dispersion stock solutions using the absorbance at 632 nm and an extinction coefficient of ∈₆₃₂=0.036 (mg/L)⁻¹ cm⁻¹.

SWCNT near-Infrared Fluorescence Measurements. SWCNT stock solutions were diluted to a concentration of 0.5 mg/L in solutions of varying pH. These solutions were incubated at room temperature overnight to allow the systems to reach equilibrium prior to collecting fluorescence and/or absorbance measurements. Fluorescence measurements were conducted in triplicate in 96-well plates (Tissue Culture Plates, Olympus Plastics) using volumes of approximately 200 μL. SWCNT solutions were excited using a 785 nm diode laser (Invictus, Kaiser Optical Systems, MI), and a 20×/0.4 N.A. objective LD Plan Neofluar (Zeiss, Germany), and inverted microscope (Zeiss AxioVision). PL was collected using the same objective with using the same gratings and detector as above. Exposure time was held constant across was 60 sec to have significant signal to noise. In all cases, fluorescence spectra were background corrected using SWCNT-free solution in an equivalent volume. During experiments in which an analyte was added, 2 μL of analyte solution was added to each well for the desired concentration and mixed on a rocking shaker for 1 hr incubation at room temperature prior to collecting fluorescence measurements. Separate wells were designated as analyte-free controls.

PCA and HLF Analysis. PCA was performed using the standard package in MATLAB via the SVD method (Natick, MA). Each HLF was first normalized by both the mean and standard deviation prior to PCA. Part of the HLF features were extracted using the oligoprop function in MATLAB.

### Sensor Response Function—Dependent Variable

The combined sensor response function is provided herein. The first term of the sensor response is designed to capture intensity modulations, by summing the normalized intensity change at each wavelength between the experimental and control was calculated. The second term is meant to capture total wavelength modulations or peak shifts, through a term describing the overall shape change of the same intensity change vector was calculated. The second term (the shape term) is designed to be small when either one of the following conditions hold, based on a manual review of the noise typical of DNA-SWCNT sensor responses:
The sensor response curve intensity is shifted up or down proportionally at all points (i.e., c(λ)=b*f(λ), ∀λ, where b∈R)
The highest peak of the sensor response curve, and the rest of the curve, are each shifted up or down proportionately at all points, but by different amounts (i.e., c(λ*)=b₁*f(λ*) for λ*=arg max_{λ}(c(λ)), but c(λ)=b₂*f(λ), ∀λ≠λ*, where b1; b2∈R)

The shape term accomplishes this by summing at each wavelength the difference in proportion between intensity for the wavelength and the highest peak of the analyte and response curves. This is accomplished with the g(λ) term. The subtraction of the term ensures that when the highest peak of the curve is the same intensity, but the rest of the curve is shifted down proportionately at all points (i.e., c(λ*)=f(λ*) for λ*=arg max_{λ}(c(λ)), but c(λ)=b*f(λ), ∀λ≠λ*, where b∈R), that β=g(λ), ∀λ≠λ*.

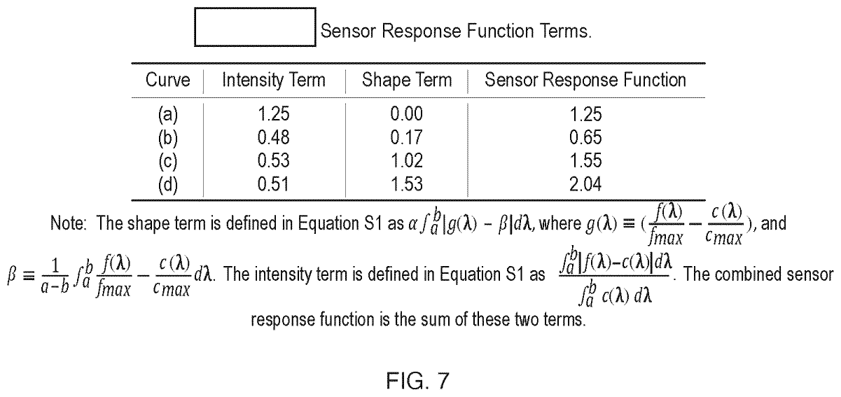

FIG. 13 shows sensor response curves for four cases. In case (a), the sensor response curve is all shifted upwards proportionally, in case (b) the peak shifts up while the rest of the curve stays the same, in case (c) the peak stays the same while the rest of the curve shifts down, in case (d) the shape of the entire response curve is more visibly different than the rest, where the first and third peaks are lower while the second and fourth are higher. Intuitively, the shape term of the sensor response function may be wanted to be larger for case (d) than for other cases. This is true of the sensor response function defined herein, whose values are shown in FIG. 7. As expected, the shape term is much larger for curve (d), leading to the total sensor response function to be the largest, despite the intensity term being smaller for curve (d) than for curves (a) or (b).

### Model Specification—Gradient-Boosted Decision Trees

The primary model used in this example for DNA-SWNCT predictions, selected for its good performance on machine learning problems for small-scale data, is gradient-based boosting on decision trees (GBDTs). A simplified overview of how the model is trained is as follows. For a given experimental condition (analyte/pH), let y denote the vector of sensor responses for all DNA sequences, and let X denote the matrix of features (including either HLF-PCs, or HLF-PCs in addition to the local structure CNN predictions). The model is then iteratively built with simple decision trees trained on residual errors:
1. Fit the stage 1 model (decision tree) f₁(·) on X, y, to minimize the residual sum of squares error
2. Calculate the first stage residual errors Resid₁=S−f₁(X)
3. Fit a first-stage residual decision tree g₁(·) on X, Resid₁, where the tree weights are fit to minimize the residual sum of squares error of f₁(·)+g₁(·) on X, y
4. Set f₂(·)=f₁(·)+ρ*g₁(·), where ρ is the learning rate
5. Calculate the second stage residual errors Resid₂=y−f₂(λ)
6. Fit a second-stage residual decision tree g₂(·) on X, Resid₂
7. Repeat until f_{M}(·) is trained

Where the depth of each decision tree, the number of stages M, and the learning rate ρ are model hyperparameters to be set with cross-validation.

This model is implemented using the XGBoost library. The details for this Python package can be found in the conference paper accompanying the library.

### Model Hyperparameters

This section provides additional details related to model hyperparameters, including selection methodology and the final hyperparameters used in the analysis.

Hyperparameter Selection: Convolutional Neural Networks. The CNN in this example uses the sensor response from Equation 1 as the dependent feature, and OHE of each DNA NB as the independent features. Various hyperparameters of single-layer CNNs are considered that relate to model complexity, including number of convolutional filters c_{f}∈{2, 4, 8, 16, 32, 64}, and motif sizes n∈{4, 6, 8}. Several regularization hyperparameters are also considered, including dropout d∈(0.0, 0.5) and number of training epochs nₑ∈{100, 150, 200, 250, 300, 350, 400, 450, 500}.

These CNN hyperparameters are selected out-of-sample, using Bayesian optimization implemented in Python with GPyOpt and a 70%/30% training/test split in order to minimize residual squared-error. The CNNs structure and the independent feature encoding are displayed in FIG. 2. Specifically, the following steps are taken:
1. Bayesian hyperparameter optimization is initialized with motif size n=6, number of convolutional filters c_{f}=32, dropout d=0.0, and training epochs nₑ=150
2. For a set of five trials, a 70%/30% training/test split of the DNA sequences is randomly produced, the CNN is trained using the current set of hyperparameters, and R² is calculated out-of-sample on the test dataset for various experimental conditions.
3. Based on the average R² for all previous sets of trials in Step 2, a new set of hyperparameters is proposed using Bayesian optimization. At a high-level, these hyperparameters are selected to optimize the expected positive gain in R², assuming that for unobserved sets of parameters the expected R² can be predicted based on observed points, while the variation in this measure is proportional to how far away it is from observed points.
4. Steps 2 and 3 are repeated for 20 iterations
5. The hyperparameter set achieving the highest out-of-sample R² is selected for the final model Hyperparameter Selection: Gradient-Boosted Decision Trees. The final predictions for DNA-SWCNT outcomes were made with gradient-boosted decision trees, implemented with XGBoost. Various hyperparameters are considered to control model complexity and regularization, including tree depth t_{d}∈{3, 4}, learning rate rho∈{0.01, 0.025, 0.05, 0.1}, and number of trees tₙ∈{100, 250, 500}.

Hyperparameters were then selected out-of-sample by performing a grid search. Specifically, the following steps were taken to select hyperparameters:
1. Select an experimental condition, and all corresponding DNA-SWCNT data
2. For each possible set of model hyperparameters, do the following:
3. For each DNA sequence in Step 1, a gradient-boosted decision tree is trained using the remaining DNA sequences as the training set (leave-one-out cross validation)
4. Using the predicted sensor response from Step 3, and the actual experimental sensor response, calculate the Pearson correlation coefficient for the experimental condition and hyperparameters
5. Repeat Steps 1-4 for all experimental conditions and all sets of model hyperparameters
6. For a given experimental condition, model hyperparameters are then selected to maximize the average Pearson correlation coefficient for all other analytes at the same pH (e.g., the model hyperparameters for chloramphenicol at pH 8 are selected to maximize the average Pearson correlation coefficient for enrofloxacin at pH 8, semicarbazide at pH 8, and cadmium at pH 8).

### Final Hyperparameters: Convolutional Neural Networks.

Hyperparameters for CNNs were selected using the methodology described previously in this section. The final hyperparameters selected were convolutional filters c_{f}=4, motif size n=4, dropout d=0.027 and number of training epochs nₑ=250. The small number of convolutional filters and motif sizes selected is noted, given the fairly small number of laboratory experiments for different DNA sequences. However, even these simple CNNs were shown to be able to improve statistical power in at least some cases (see the results section in the example).

### Final Hyperparameters: Gradient-Boosted Decision Trees.

Hyperparameters for gradient-boosted decision trees were selected using the methodology described previously in this section. The final hyperparameters are shown in FIG. 8. The approach selected more sophisticated models (with a higher tree depth and lower learning rate) for the DNA-SWCNT sensors at pH 6 than at pH 8, which is consistent with the fact that the sensors exhibited greater responses at pH 6 (see the results section in the example).

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Embodiments of the present invention include features which are only possible and/or feasible to implement with the use of one or more computers, computer processors, and/or other elements of a computer system. Such features are either impossible or impractical to implement mentally and/or manually. For example, the methods for making and using computational models are only practical and/or possible to implement using a computer.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

Any step or act disclosed herein as being performed, or capable of being performed, by a computer or other machine, may be performed automatically by a computer or other machine, whether or not explicitly disclosed as such herein. A step or act that is performed automatically is performed solely by a computer or other machine, without human intervention. A step or act that is performed automatically may, for example, operate solely on inputs received from a computer or other machine, and not from a human. A step or act that is performed automatically may, for example, be initiated by a signal received from a computer or other machine, and not from a human. A step or act that is performed automatically may, for example, provide output to a computer or other machine, and not to a human.

The terms “A or B,” “at least one of A or/and B,” “at least one of A and B,” “at least one of A or B,” or “one or more of A or/and B” used in the various embodiments of the present disclosure include any and all combinations of words enumerated with it. For example, “A or B,” “at least one of A and B” or “at least one of A or B” may mean: (1) including at least one A, (2) including at least one B, (3) including either A or B, or (4) including both at least one A and at least one B.

1. A method, performed by at least one computer processor, of making a computational model stored on at least one non-transitory computer-readable medium, the method comprising:
selecting a candidate molecular recognition (MR) binder that comprises a nanomaterial, wherein the nanomaterial is configured for signal transduction in response to a molecular recognition event that involves the candidate MR binder;
recording an experimental result that corresponds with a physical test of the candidate MR binder; and
correlating a feature of the candidate MR binder with the experimental result to produce predictive information that is embodied by the computational model.

2. The method of claim 1, wherein the method is performed by the at least one computer processor executing computer readable instructions stored on the at least one non-transitory computer-readable medium.

3. The method of claim 1, wherein the experimental result indicates whether or not the molecular recognition event occurred in the physical test, wherein the molecular recognition event involves a physical interaction between the candidate MR binder and an analyte.

4. The method of claim 1, wherein the experimental result indicates a degree to which the molecular recognition event occurred in the physical test, wherein the molecular recognition event involves a physical interaction between the candidate MR binder and an analyte.

5. The method of claim 1, wherein the nanomaterial comprises a single-walled carbon nanotube (SWCNT) that is optically responsive to the molecular recognition event.

6. The method of claim 1, wherein the candidate MR binder comprises an interaction feature for interaction with an analyte for the molecular recognition event, wherein the interaction feature comprises an aptamer, a polymer, a peptide, a polypeptide, a protein, a protein complex, a ribonucleic acid (RNA), or any combination thereof.

7. The method of claim 1, wherein an interaction feature of the candidate MR binder comprises a polynucleotide for interaction with an analyte for the molecular recognition event.

8. The method of claim 7, wherein the polynucleotide comprises deoxyribonucleic acid (DNA).

9. The method of claim 7, wherein the analyte is selected from the group consisting of: cadmium, enrofloxacin, chloramphenicol, semicarbazide, and any combination thereof.

10. The method of claim 7, wherein correlating the feature comprises:
analyzing, with a convolutional neural network (CNN), a local structure of DNA as the feature to make a local structure prediction (LSP).

11. The method of claim 7, wherein correlating the feature comprises:
analyzing, with a principal components analysis (PCA), a high-level feature (HLF) of DNA as the feature to make a high-level prediction (HLP).

12. The method of claim 7, wherein correlating the feature comprises:
analyzing, with a convolutional neural network (CNN), a local structure of DNA as a first feature to make a local structure prediction (LSP);
analyzing, with a principal components analysis (PCA), a high-level feature (HLF) of DNA as a second feature to make a high-level prediction (HLP); and
analyzing, with a gradient-boosted decision tree (GBDT), the LSP and the HLP to make predictive information that relates the first feature and the second feature to the experimental result.

13. A method, performed by at least one computer processor, of using a computational model stored on at least one non-transitory computer-readable medium, the method comprising:
receiving, as an input, a feature of a candidate molecular recognition (MR) binder that comprises a nanomaterial, wherein the nanomaterial is configured for signal transduction in response to a molecular recognition event that involves the candidate MR binder;
analyzing the feature of the candidate MR binder, based on the computational model, to produce a prediction score that represents an expected experimental result of a physical test of the candidate MR binder; and
associating the prediction score with the candidate MR binder.

14. The method of claim 13, wherein the method is performed by the at least one computer processor executing computer readable instructions stored on the at least one non-transitory computer-readable medium.

15. The method of claim 13, wherein the expected experimental result indicates whether or not the molecular recognition event is expected to occur in the physical test, wherein the molecular recognition event involves a physical interaction between the candidate MR binder and an analyte.

16. The method of claim 13, wherein the expected experimental result indicates a degree to which the molecular recognition event is expected to occur in the physical test, wherein the molecular recognition event involves a physical interaction between the candidate MR binder and an analyte.

17. The method of claim 13, wherein the nanomaterial comprises a single-walled carbon nanotube (SWCNT) that is optically responsive to the molecular recognition event.

18. The method of claim 13, wherein the candidate MR binder comprises an interaction feature for interaction with an analyte for the molecular recognition event, wherein the interaction feature comprises an aptamer, a polymer, a peptide, a polypeptide, a protein, a protein complex, a ribonucleic acid (RNA), or any combination thereof.

19. The method of claim 13, wherein an interaction feature of the candidate MR binder comprises a polynucleotide for interaction with an analyte for the molecular recognition event.

20. The method of claim 19, wherein the polynucleotide comprises deoxyribonucleic acid (DNA).

21. The method of claim 19, wherein the analyte is selected from the group consisting of: cadmium, enrofloxacin, chloramphenicol, semicarbazide, and any combination thereof.

22. The method of claim 19, wherein analyzing the feature comprises:
analyzing, with a convolutional neural network (CNN), a local structure of DNA as the feature to make a local structure prediction (LSP).

23. The method of claim 19, wherein analyzing the feature comprises:
analyzing, with a principal components analysis (PCA), a high-level feature (HLF) of DNA as the feature to make a high-level prediction (HLP).

24. The method of claim 19, wherein analyzing the feature comprises:
analyzing, with a convolutional neural network (CNN), a local structure of DNA as a first feature to make a local structure prediction (LSP);
analyzing, with a principal components analysis (PCA), a high-level feature (HLF) of DNA as a second feature to make a high-level prediction (HLP); and
analyzing, with a gradient-boosted decision tree (GBDT), the LSP and the HLP to make the prediction score that relates the first feature and the second feature to the expected experimental result.

25. A non-transitory computer-readable medium having stored thereon the computational model of claim 13.

26. A computer system configured to perform the method of claim 1.
